(19) United States
(12) Patent Application Publication
(10) Pub. No.: US 2026/0199494 A1
(43) Pub. Date: Jul. 16, 2026

(54) BIVALENT COVALENT PROTEIN-BINDING MOLECULES, METHODS OF MAKING AND USES THEREOF

(71) Applicant: McMaster University, Hamilton (CA)

(72) Inventors: Anthony Rullo, Hamilton (CA); Eden Kapcan, Hamilton (CA); Karolina Krygier, Calgary (CA); Maya Da Luz, Toronto (CA)

(21) Appl. No.: 19/421,711

(22) Filed: Dec. 16, 2025

Related U.S. Application Data
(60) Provisional application No. 63/734,435, filed on Dec. 16, 2024.

(51) Int. Cl. A61K 47/64; A61P 35/00

(52) U.S. Cl. CPC A61K 47/646; A61K 47/6425; A61P 35/00

(57) ABSTRACT

This application describes bivalent covalent glue mimics (CGMs). More particularly, the present application relates to protein-binding molecules that induce the formation of stable ternary complexes via covalent binding to two proteins (i.e. selective chemical crosslinking), and methods of making and uses thereof. Accordingly, included is a compound of Formula (I), Formula (II) or Formula (III) or a pharmaceutically acceptable salt and/or solvate thereof, or a composition comprising the same.

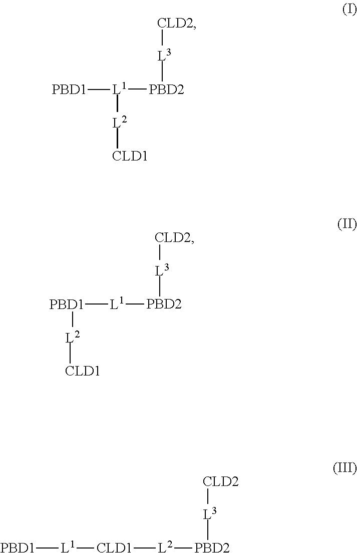

### CROSS REFERENCE TO RELATED APPLICATION

The present application claims the benefit of priority from U.S. provisional application No. 63/734,435 filed on Dec. 16, 2024, the contents of which are incorporated herein by reference in their entirety.

### INCORPORATION OF SEQUENCE LISTING

A computer readable form of the Sequence Listing “P93249844US02_96640724_SequenceListing.xml” (20,480 bytes), filed herewith by electronic submission and created on Dec. 16, 2025, is herein incorporated by reference.

### FIELD

The present application relates to bivalent covalent glue mimics (CGMs). More particularly, the present application relates to protein-binding molecules that induce the formation of stable ternary complexes via covalent binding to two proteins (i.e. selective chemical crosslinking), and methods of making and uses thereof.

### BACKGROUND

Proximity inducing molecules (PIMs) represent a growing class of novel therapeutic modalities, with special utility across a diverse range of disease indications.^{1,2} PIMs generally increase the effective molarity between two proteins in a ternary complex to enact a biological response. A special class of PIMs called molecule glues (MGs) leverage positive co-operativity to drive and stabilize ternary complex formation enhancing this response.³⁻⁵ Here, molecular glue binding to both proteins is more thermodynamically favorable than binding to either protein alone, which drives ternary complex formation in the forward direction. A key example is the anti-cancer drug rapamycin, which templates inhibitory ternary complexes between FKBP and the kinase mTOR, the latter a key regulator of tumor growth and proliferation. Here, rapamycin binding to FKBP first, dramatically increases rapamycin apparent affinity for mTOR (and vice versa) due to stabilizing protein-protein interactions.

Stabilizing ternary complexes is especially strategic when endogenous species concentrations are low relative to their K_{d}, for PIM binding and/or when competitive protein binding ligands are present. The discovery and development of PIMs with glue-like properties is challenging however, especially for applications that aim to bridge non-interacting proteins or proteins that interact unfavorably within the ternary complex, the latter associated with negative co-operativity.^{6,7} These scenarios are likely to be encountered when bridging receptors on the surfaces of two cells to induce “cell-cell proximity”. Fundamental efforts in tumor immunotherapy leverage bispecific biologic or small molecule PIM modalities to induce cell-cell proximity. One key example uses antibody recruiting molecules to enhance immune cell recognition and elimination of tumor cells.^{8,9} Here the PIM must form stable ternary complexes comprising a tumor antigen and serum antibody. Immune cell Fc receptors subsequently engage these ternary complexes leading to tumor eradication. In addition to steric clashing between tumor and immune cells, immune activation via Fc receptor clustering/synapse formation proceeds through mechanical re-arrangement events. Collectively, these forces can destabilize even thermodynamically favorable protein complexes as observed in the case of “slip bonds”.^{10,11} Thus, the ability to even partially stabilize cell-cell proximity in a “glue-like” manner is highly strategic.

There is a need to develop new proximity inducing molecules (or covalent glue mimics (CGMs)) providing, for example, high binding affinity for target, increased stability of immunological synapses and/or lower pharmacokinetic clearance.

The background herein is included solely to explain the context of the application. This is not to be taken as an admission that any of the material referred to was published, known, or part of the common general knowledge as of the priority date.

### SUMMARY

A bifunctional molecule-dual proximity labeling strategy to irreversibly covalently crosslink two proteins, mimicking molecular glue stabilization has been developed and is disclosed herein. The bivalent covalent proximity inducing molecules, or covalent glue mimics (CGMs), of the present application induced protein-protein interactions that led to cell-cell proximity in three distinct tumor immunotherapeutic model systems, leading to significant functional enhancements. Collectively, this underscores the utility of irreversible dual proximal covalent labeling to stabilize and enforce protein-protein interactions, including those on the surface of cells allowing for cell-cell interactions.

Accordingly, the present application includes a compound of Formula (I), Formula (II) or Formula (III) or a pharmaceutically acceptable salt, and/or solvate thereof, wherein PBD1 and PBD2 are each independently a protein binding domain (PBD) and CLD1 and CLD2 are each independently a covalent labeling domain (CLD), and L1, L2 and L3 are each independently a linker group;
wherein PBD1 selectively binds to a first target protein and PBD2 selectively binds to a second target protein, and wherein CLD1 comprises a functional group that, upon binding of PBD1 to the first target protein, forms an irreversible covalent bond with a nucleophilic group in the first target protein and CLD2 comprises a functional group that, upon binding of PBD2 to the second target protein, forms an irreversible covalent bond with a nucleophilic group in the second target protein.

Also included is a composition comprising the compound of Formula (I), Formula (II) or Formula (III), or a pharmaceutically acceptable salt and/or solvate thereof, and at least one carrier, diluent and/or excipient.

The present application further includes a method for forming ternary protein complexes, either in a biological sample or in a subject, comprising administering an effective amount of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, to the biological sample or subject.

Also included is a method for recruiting an antibody or an immune cell for immunotherapy, either in a biological sample or in a subject, comprising administering an effective amount of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, to the biological sample or subject.

The present application further includes a method for recruiting an antibody or an immune cell and targeting a cell for provoking an immune response to the cell, either in a biological sample or in a subject, comprising administering an effective amount of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, to the biological sample or the subject.

The present application includes a method for binding tumor antigens on a cell, either in a biological sample or in a subject, comprising administering an effective amount of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, to the biological sample or the subject.

Further included is a method of treating a disease, disorder, or condition that is treatable by engaging an immune response, comprising administering a therapeutically effective amount of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, to a subject in need thereof.

The present application includes use of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, for forming ternary protein complexes, either in a biological sample or in a subject.

The present application further includes a use of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, for forming ternary protein complexes, either in a biological sample or in a subject.

Also included is use of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, for recruiting an antibody or an immune cell for immunotherapy, either in a biological sample or in a subject.

Further included is use of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, for recruiting an antibody or an immune cell and targeting a cell for provoking an immune response to the cell, either in a biological sample or in a subject.

The present application further includes use of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, for binding tumor antigens on a cell, either in a biological sample or in a subject.

The present application also includes use of a compound of Formula (I), Formula (II) or Formula (III), or a pharmaceutically acceptable salt and/or solvate thereof, or a composition comprising a compound of Formula (I), Formula (II) or Formula (III), or a pharmaceutically acceptable salt and/or solvate thereof, for treating a disease, disorder, or condition that is treatable by engaging an immune response.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

Certain embodiments of the application will now be described in greater detail with reference to the attached drawings in which:

FIG. 1A and FIG. 1B shows a schematic depiction of molecular glues versus covalent glue mimics in exemplary embodiments of the application: FIG. 1A molecular glues (MGs) use positive cooperativity to stabilize a ternary complex thermodynamically; FIG. 1B covalent glue mimics (CGMs) use strategically positioned dual covalent labeling electrophiles to form kinetically irreversible ternary complexes.

FIG. 2 shows a comparison between AE105-DNP and bifunctional molecule (BM) ternary complex formation, in which AE105-DNP is unable to mediate effective ternary complex between uPAR functionalized on the probe and anti-DNP (lighter trace) while BM containing a “GSGG” linker between the DNP and uPAR binding moieties according to an exemplary embodiment of the application, is able to mediate effective ternary complex as demonstrated by association and dissociation in the BLI spectrogram (black trace).

FIG. 3 shows the chemical strategy to control electrophile insertion position on bifunctional molecules in exemplary embodiments of the application: peptides were synthesized using Standard Fmoc SPPS chemistry using reaction conditions A) 0.2M 3-fluorosulfonyl benzoic acid, IM DIC, IM Oxyma, 2 minutes at 90 degrees; A′) 10% Acetic Anhydride, 10 minutes at RT; B) 2% Hydrazine in DMF, 2×15 minutes at RT; C) 0.2M 3-fluorosulfonyl benzoic acid, IM DIC, IM Oxyma, 2 minutes at 90 degrees; D) 95:2.5:2.5 TFA: H2O: TIPS for 3 hours at RT.

FIG. 4 shows validation of cBMs using biosensor assays in exemplary embodiments of the application: panel A) BM and cBM chemical structures, in which covalent bifunctional molecule “^{Ab}cBM” is designed to selectivity react with anti-DNP, covalent bifunctional molecule “^{uPAR}cBM”, is designed to selectivity react with uPAR, and analogs ^{uPAR}cBM (2-4) were informed by uPAR: AE133 analogue co-crystal structures to enhance uPAR labeling kinetics; panel B) schematic of BLI assays performed using the steps of: Incubation: cBM is incubated with biotinylated terminal Protein 1; Load: the incubation solution is loaded onto a streptavidin biosensor; Wash: Probes are submerged in buffer wells to remove non-covalent complex; and Association: Probes are submerged in a solution containing soluble terminal Protein 2, giving rise to association signal proportional to covalent reaction; panel C) ^{Ab}cBM incubation with anti-DNP for different periods of time (terminal Protein 1=anti-DNP, terminal Protein 2=uPAR); panel D) uPAR-cBM incubation with uPAR for different periods of time (terminal Protein 1=uPAR, terminal Protein 2=anti-DNP); panel E) repeat of the experiment in FIG. 4D using ^{uPAR}cBM derivatives; panel F) intra ternary complex selectivity assay swapping Protein 1 and Protein 2 for each compound during the incubation step—i.e. ^{Ab}cBM (terminal Protein 1=uPAR, terminal Protein 2=anti-DNP), ^{uPAR}cBM (terminal Protein 1=anti-DNP, terminal Protein 2=uPAR)—a zero association signal indicates complete intra ternary complex selectivity with a cartoon depiction of the undesired competitive labeling reaction between ^{uPAR}cBM-2 and anti-DNP versus the intended uPAR.

FIG. 5A and FIG. 5B show ^{Ab}cBM and ^{uPAR}cBM selectivity controls in exemplary embodiments of the application: FIG. 5A spectrogram of ^{uPAR}cBM+BSA and Isotype Ab, leading to baseline signal and FIG. 5B spectrogram of ^{Ab}cBM+BSA and Isotype Ab, leading to baseline signal.

FIG. 6A and FIG. 6B show plots of fraction reaction conversion as a function of incubation time to evaluate ^{Ab}cBM and ^{uPAR}cBM labeling kinetics in exemplary embodiments of the application: FIG. 6A is a plot of fraction covalently labeled antibody vs time to determine CGM-Ab labeling kinetics, t1/2~ 0.16 h; and FIG. 6B is a plot of fraction covalently labeled uPAR vs time to determine kinetics of CGM-uPAR, t12~ 2.5 h-fraction reaction conversion was determined by dividing the associations plateau signal at each reaction time point, with the maximal plateau signal assigned to zero free unreacted protein remaining, generated at reaction end point in FIG. 4, panel C and panel D; data was fit using GraphPad Prism with the first order association equation to obtain kobs.

FIG. 7A and FIG. 7B shows MALDI confirmation of covalent binary complexes in exemplary embodiments of the application: FIG. 7A is MALDI analysis of ^{Ab}cBM incubation with anti-DNP Ab, in which Ab alone corresponds to a peak centered at m/z=148322 And Ab incubated with CGM-Ab gives rise to a peak centered at m/z=152216—the 3894 Da difference (Expected mass=3892) corresponds to quantitative antibody labeling with two Ab^{cBM} molecules linking to one anti-DNP molecule (one per Fab) minus an equivalent of HF; and FIG. 7B is MALDI analysis of ^{uPAR}cBM incubation with uPAR, in which uPAR alone corresponds to a peak centered at m/z=41541 and uPAR incubated with ^{uPAR}cBM corresponds to a peak centered at m/z=42628—the 1087 Da difference corresponds to approximately 53% uPAR labeling—and uPAR incubated with ^{uPAR}cBM2 corresponds to a peak centered at m/z=42928—the 1387 Da difference corresponds to approximately 93% uPAR labeled.

FIG. 8A, FIG. 8B and FIG. 8C show labeling kinetics measurements of ^{uPAR}cBM and ^{Ab}cBM using fluorescent SDS-PAGE in exemplary embodiments of the application: FIG. 8A is gel image of uPAR labeling kinetics monitored over time via fluorescent SDS-PAGE using Cy5 channel (top) and plot of increase in fluorescence band intensity with time used to estimate the half-life for ^{uPAR}cBM labeling of uPAR of t1/2~ 1.5h (bottom); FIG. 8B is gel image of anti-DNP labeling kinetics monitored over time via fluorescent SDS-PAGE using Cy5 channel (top) and plot of fraction reacted vs time to determine kinetics of ^{uPAR}cBM to yield t1/2~ 0.4 h (bottom); and FIG. 8C is anti-DNP and uPAR reaction selectivity monitoring via fluorescent SDS-PAGE in exemplary embodiments of the application: Coomassie staining of anti-DNP Ab incubated with CGM-Azide and DNP-Glycine competitor for various times, 0-2h (top left); fluorescence channel (Cy5) showing no labeling with AE133 competitor, indicating selectivity (top right); Coomassie staining of uPAR incubated with CGM-Azide and AE133 competitor for various times, 0-24h (bottom left); fluorescence channel (Cy5) showing no labeling with AE133 competitor, indicating selectivity (bottom right).

FIG. 9 shows BLI assays of CGM templating ‘infinitely stable’ irreversible ternary complexes in exemplary embodiments of the application: panel A) chemical structure of CGM; panel B) schematic of BLI assay procedure to evaluate covalent crosslinking within ternary complexes: Incubation: CGM is incubated with both biotinylated uPAR and anti-DNP; Load: this solution is loaded onto a streptavidin biosensor probe; Two-Step Competitor Wash: the probe is submerged in a solution of free uPAR competitor (first wash step) and then placed in a solution of free DNP competitor to complex anti-DNP (second wash step); panel C) BLI spectrogram from covalent ternary complex assays evaluating CGM versus ^{uPAR}cBM, ^{Ab}cBM or BM; panel D) orthogonal SDS-PAGE assay to probe covalently crosslinked ternary complex formation, using Coomassie protein staining; panel E) scatter plot generated from a flow cytometry antibody recruiting assay, in which uPAR+A172 cells were incubated with CGM or BM and Anti-DNP Ab-AF647 for 4 hours at 37° C., followed by wash steps-gates were placed on the antibody alone condition.

FIG. 10 shows labeling kinetics measurements of CGM using SDS-PAGE in exemplary embodiments of the application: SDS-PAGE gel time course in which covalent ternary complex formation via CGM was monitored by the appearance of a new larger molecular weight band (Coomassie) not observed using cBMs (top) and density vs time plot to estimate the kinetics of CGM mediated ternary complex crosslinking-using Prism GraphPad the curve was fitted to a one phase association equation to yield t1/2~ 2.5h (bottom).

FIG. 11 shows CGM elicits greater anti-tumor blockade and immunotherapeutic function compared to mono-covalent bifunctionals in exemplary embodiments of the application: panel A) schematic depiction of the microscopy uPA-uPAR competition assay in which uPa-AF647 alone leads to an increase in immunofluorescence and incubation with CGM or BM leads to decreases in immunofluorescence; panel B) immunofluorescence microscopy images depicting CGM versus BM mediated uPA blockade-cells only (top left); 25 nM uPA-AF647 for 15 minutes (top right); 100 nM BM for 1 hour, followed by 25 nM uPA-AF647 for 15 minutes (bottom left); 100 nM CGM for 1 hour, followed by 25 nM uPA-AF647 for 15 minutes (bottom right); panel C) antibody dependent mediated phagocytosis (ADCP) assay in which uPAR-Biotin, bifunctional molecules, and anti-DNP Ab are incubated overnight and the next day, this solution is added to fluorescent streptavidin microspheres to model cancer targets+fluorescent u937 cultured macrophage immune cells for 1 hour-two color flow cytometry is used to measure phagocytosis; panel D) CD16a activation ADCC assay in which CD16+Jurkat T-cell lines are incubated with uPAR+A172 cells, bifunctional compounds, and antibody and after 24 hours luciferase-substrate is added to each well, luminescence is measured, and correlated to immune activation.

FIG. 12A and FIG. 12B show ADCP assays of CGM in exemplary embodiments of the application: FIG. 12A is representative flow cytometry scatterplot associated with the 50 nM CGM condition; and FIG. 12B is a bar graph representation of ADCP data with control conditions.

FIG. 13 shows model NK cell CD16 activation control experiments in exemplary embodiments of the application.

FIG. 14 shows a cell cytotoxicity assay of CGMs inducing cell-cell proximity activating tumoricidal T cell function in exemplary embodiments of the application: panel A) cytotoxicity assay using live cell microscopy monitoring A172 tumor cell death in SAR-T cell co-cultures as a function of time over Day 0 (left), Day 1 (middle) and Day 2 (right) using uPAR+A172s (light grey) and SAR T-Cells (dark grey) with BM (100 nM; top row), ^{Ab}cBM (1 nM, second row), ^{uPAR}cBM (1 nM, third row) and CGM (1 nM, fourth row); panel B) Day 2, InM CGM condition, magnified, showing high density of SAR T-cells illustrating T cell proliferation and reduced tumor cell count; panel C) bar graph depiction of T cell cytotoxicity quantification; D) SAR T-cell cytotoxicity time course using Green Image Mean.

FIG. 15A, FIG. 15B, and FIG. 15C shows proliferation assays using bifunctional molecule-induced T cell proliferation in exemplary embodiments of the application: FIG. 15A is a representative gating strategy for analyzing proliferation samples with cell trace violet (CTV) traces fit using the proliferation fit statistics analysis software from FCS express; FIG. 15B is a CTV dilution traces using T cells incubated with media alone and tumors without adapters as controls—a decrease in CTV MFI upon compound treatment indicates increased proliferation;

FIG. 15C is plot of number of cells which entered division at concentrations of InM, 10 nM and 100 nM of each construct (calculated using the proliferation fit statistics)—showing CD8 T cells as both CD4 and CD8 T cells displayed similar levels of division (bottom).

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art. It is also to be understood that the terminology used herein is for the purpose of describing particular aspects only and is not intended to be limiting.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least±5% of the modified term if this deviation would not negate the meaning of the word it modifies. In addition, all ranges given herein include the end of the ranges and also any intermediate range points, whether explicitly stated or not.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.”

The word “or” is intended to include “and” unless the context clearly indicates otherwise.

The term “compound(s) of the application” or “compound(s) of the present application” and the like as used herein refers to a compound of Formula (I), (II) or (III) or pharmaceutically acceptable salts and/or solvates thereof.

The term “composition(s) of the application” or “composition(s) of the present application” and the like as used herein refers to a composition, such a pharmaceutical composition, comprising one or more compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means a bivalent straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “aryl” as used herein, whether it is used alone or as part of another group, refers to carbocyclic groups containing at least one aromatic ring and contains 6 to 20 carbon atoms.

The term “amine” or “amino,” as used herein, whether it is used alone or as part of another group, refers to groups of the general formula NR′R″, wherein R′ and R″ are each independently selected from hydrogen or C₁₋₆alkyl.

The term “amino acid” as used herein refers to an organic compound comprising amine (—NH₂) and carboxylic acid (—COOH) functional groups, along with a side-chain specific to each amino acid. The common elements of an amino acid are carbon, hydrogen, oxygen and nitrogen, though other elements are found in the side-chains of certain amino acids, including S and Se. Unless otherwise specified, an amino acid referenced herein is one of the 23 proteinogenic amino acids, that is amino acids that are precursors to proteins, and are incorporated into proteins during translation.

The following symbol:

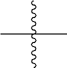

is used in chemical structures herein to represent a point of covalent attachment of a group to another group.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3rd Edition, 1999 and in Kocienski, P. Protecting Groups, 3rd Edition, 2003, Georg Thieme Verlag (The Americas).

The term “linker” or “linker group” as used herein refers to any molecular structure that joins two or more other molecular structures together and that is compatible with a biological environment.

The term “compatible with a biological environment” as used herein it is meant that the chemical group or molecule is stable in, and/or does not denature, other molecules present in biological systems.

The term “biological systems” as used herein means any of a wide variety of systems which comprise proteins, enzymes, organic compounds, inorganic compounds, other sensitive biopolymers including DNA and RNA, and includes complex systems such as whole or fragments of plant, animal and microbial cells.

The term “subject” as used herein includes all members of the animal kingdom including mammals, and suitably refers to humans. Thus, the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

The term “treats”, “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The term “immunotherapy” as used herein refers to the treatment of disease, disorder or condition by activating the immune system to produce or provoke an immune response.

The term “immune response” as used herein refers to the activation of immune cells.

The term “hapten” as used herein refers to a small molecule that can elicit an immune response only when attached to a large carrier such as a protein. The carrier may be one that also does not elicit an immune response by itself.

As used herein, the term “effective amount” or “therapeutically effective amount” means an amount of one or more compounds of the application that is effective, at dosages and for periods of time necessary to achieve the desired result. For example, in the context of treating a disease, disorder or condition, an effective amount is an amount that, for example, treats the disease, disorder or condition compared to without administration of the one or more compounds.

The term “administered” as used herein means administration of a therapeutically effective amount of one or more compounds or compositions of the application to a cell, tissue, organ or subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder.

The term “cancer” as used herein refers to cellular-proliferative disease states.

The term “irreversible” as used herein refers to a covalent bond between two entities that cannot degrade on biologically relevant timescales and/or degradation is slower than the degradation of the one entity forming the covalent bond.

The term “selective” as used herein having regard to chemical entities means that an entity is more likely to bind to a target site with little or no detectable binding to non-target sites over a particular time period.

It will be understood that any component defined herein as being included may be explicitly excluded by way of proviso or negative limitation, such as any specific compounds or method steps, whether implicitly or explicitly defined herein.

### II. Compounds and Compositions of the Application

The lack of available molecular glues to stabilize cell-cell proximity inspired the efforts disclosed herein for devising a strategy that “kinetically” mimics molecular glue stabilization using covalency. This was accomplished using chimeric molecules that irreversibly crosslink both protein binding partners of the ternary complex, preventing dissociation, by using chimera that both selectively discriminate between the two ternary complex proteins, and also preferentially crosslink these proteins versus abundant off-target proteins. This was achieved by functionalizing the chimera at two optimal positions with pre-organized electrophiles, each activated upon binding to its cognate ternary complex binding partner. The strategy leverages proximal labeling-kinetic effective molarity enhancements to enforce selective crosslinking, while providing access to diverse endogenous proteins. The above is illustrated in FIG. 1A and FIG. 1B, where FIG. 1A shows a schematic depiction of molecular glues (MGs) use of positive cooperativity to stabilize a ternary complex thermodynamically, and FIG. 1B shows a schematic depiction of covalent glue mimics (CGMs) use of strategically positioned dual covalent labeling electrophiles to form kinetically irreversible ternary complexes.

Thus, disclosed herein is the development of bivalent proximity inducing molecules (PIMs), or covalent glue mimics (CGMs), that selectively and irreversibly covalently cross link at least two proteins to form a ternary complex, such as two non-interacting endogenous proteins in cell surface ternary complexes. In the context of immunotherapeutic model systems of cell-cell induced proximity, CGMs were designed to crosslink serum antibodies or immune cell receptors, with the uPAR tumor antigen on the surface of cancer cells as an exemplary embodiment. In both macrophage and NK cell functional immunotherapeutic assays, it was demonstrated that the irreversible covalent crosslinking of antibody with uPAR via CGM enables significant functional enhancements, compared to covalent engagement of either antibody or tumor antigen alone. Stable engagement of CGMs with the tumor cell surface was observed to strategically prevent competition from urokinase, an ultra-high affinity endogenous competitor ligand for uPAR, whose binding drives cancer metastasis. Additionally, it is shown that CGMs can induce universal synthetic T cell-tumor cell proximity and enact T cell tumoricidal function.

Herein it is demonstrated that the pre-organization of two electrophiles on a single bifunctional molecule can selectively and irreversibly crosslink two non-interacting proteins, stabilizing the ternary complex. Collectively, this application describes an irreversible bicovalent proximity inducing molecule with therapeutic potential, and underscores their utility for driving biomolecular interactions that lack positive co-operativity.

Accordingly, included in the present application is a compound of Formula (I), Formula (II) or Formula (III), or a pharmaceutically acceptable salt, and/or solvate thereof,

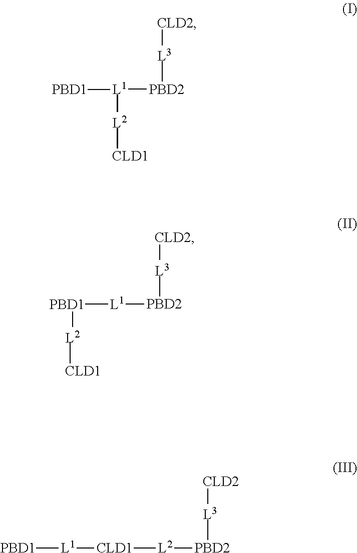

wherein PBD1 and PBD2 are each independently a protein binding domain (PBD) and CLD1 and CLD2 are each independently a covalent labeling domain (CLD), wherein PBD1 selectively binds to a first target protein and PBD2 selectively binds to a second target protein, and wherein CLD1 comprises a functional group that, upon binding of PBD1 to the first target protein, forms an irreversible covalent bond with a nucleophilic group in the first target protein and CLD2 comprises a functional group that, upon binding of PBD2 to the second target protein, forms an irreversible covalent bond with a nucleophilic group in the second target protein.

### Protein Binding Domains (PBD)

Protein Binding Domains (PBDs) of the present application are chemical entities comprising moieties which bind to target proteins to bring the proteins in proximity, intended to irreversibly covalently crosslink two proteins within ternary complexes, mimicking molecular glue stabilization. In some embodiments, the PBDs of the application will each be selected to bind a target protein so that induced proximity will generate a beneficial effect. For example, the induced cell-cell proximity in three distinct tumor immunotherapeutic model systems (Macrophage based, NK cell based, and T cell based), leading to significant functional enhancements may be provided. In some embodiments, one of the PBDs will bind to a target protein such that it will be retained in a particular site of a subject such as a biological structure for example an organ or tissue or a pathological structure for example a tumor, with little or no detectable accumulation and/or retention in non-target sites over a particular time period, while the other PBD will bind to a protein that has a benefit when brought in proximity of the organ, tissue or pathological structure.

Known uses of molecular glues include bringing together two proteins to allow for protein degradation (by hijacking the ubiquitin-proteasome system) for example to degrade known disease causing or driving proteins such as oncogenic proteins, to increase protein stabilization, to target cells for immunotherapy, among other known uses.

In some embodiments, one of the PBD binds to a target protein, for example a protein that is overexpressed in a disease, disorder or condition such as cancer and the other PBD binds to a target protein that acts as an effector to affect a beneficial effect including, without limitation, protein degradation, protein stabilization, cytotoxicity, phagocytosis and apoptosis and other immunological phenomena. Target proteins and their PBDs are known and the selection of suitable PBDs for a particular therapeutic use can be made by a person skilled in the art. PBDs include, but are not limited to, small molecules such as protein binding compounds, enzyme inhibitors or pharmaceutical-like compounds.

In some embodiments, a first PBD binds to a synthetic antigen receptor on the surface of an immune cell, resulting in a primed immune cell that, via the second PBD, targets a protein on the cell of interest. Synthetic antigen receptors may comprise an acceptor moiety that is bound by the first PBD, and an effector domain which effects signaling in an engineered cell. Exemplary synthetic antigen receptors are described in WO2023/230729, incorporated herein by reference.

In some embodiments, PBD1 and PBD2, are each different and independently comprise a moiety that binds to antigens on the surface of a target cell. In some embodiments, one of PBD1 and PBD2 comprises a moiety that binds to antigens on the surface of a target cell and the other of PBD1 and PBD2 comprises a moiety that binds to an immune effector. In some embodiments, one of PBD1 and PBD2 comprises a moiety that binds to proteins on the surface of a target cell and the other of PBD1 and PBD2 comprises a moiety that binds to an antibody. In some embodiments, one of PBD1 and PBD2 is a glutamate urea ligand that binds to prostate specific membrane antigen (PSMA) and the other of PBD1 and PBD2 comprises a moiety that binds to an effector, such as an immune cell or an antibody. In some embodiments, one of PBD1 and PBD2 is selected from any of the PBD groups described in U.S. Pat. No. 9,296,708 for targeting a cell, incorporated herein by reference. Accordingly, in some embodiments, one of PBD1 and PBD2 is a domain that targets a cell and is selected from the following groups:

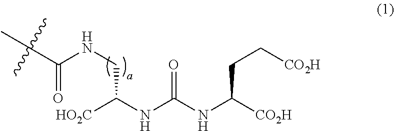

wherein a is an integer from 0 to 10, 1 to 15, 1 to 10, 1 to 8, or 1, 2, 3, 4, 5 or 6;

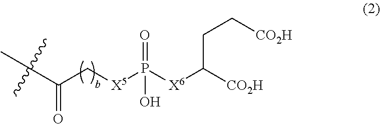

wherein X⁵ and X⁶ are independently CH₂, O, NH or S; and
b is an integer from 0 to 10, 1 to 15, 1 to 10, 1 to 8, or 1, 2, 3, 4, 5 or 6;

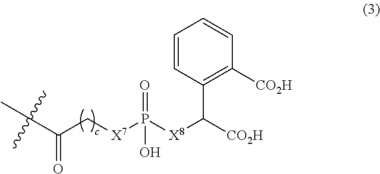

wherein X⁷ and X⁸ are independently CH₂, O, NH or S; and
c is an integer from 0 to 10, 1 to 15, 1 to 10, 1 to 8, or 1, 2, 3, 4, 5 or 6;

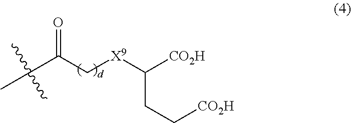

wherein X⁹ is O, CH₂, NR⁵, S(O), SO₂, SO₂O, OSO₂ or OSO₂O;
R⁵ is H, C₁-4alkyl or C(O)C₁₋₄alkyl; and
d is an integer from 0 to 10, 1 to 15, 1 to 10, 1 to 8, or 1, 2, 3, 4, 5 or 6; and
(5) biotin or a biotin analog such as:

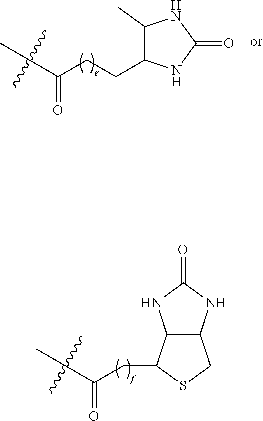

wherein e and f are, independently, an integer from 0 to 10, 1 to 15, 1 to 10, 1 to 8, or 1, 2, 3, 4, 5 or 6.

In some embodiments, a, b, c, d, e and f are independently 1, 2, 3, 4, 5 or 6, suitably 2, 3 or 4, more suitably 4.

In some embodiments, one of PBD1 and PBD2 comprise other tumor antigen binding ligands such as synthetic peptides against uPAR or HER2, or folate receptor binding molecules such as folate or methotrexate, or TLR agonists or PD-1/PD-L1 antagonists.

In some embodiments, one of PBD1 and PBD2 comprise a hapten which binds to an antibody that is endogenous in a subject. In some embodiments, the antibody is present in the subject prior to treatment (i.e. the antibody levels do not have to be raised in the subject prior to treatment). In some embodiments, the antibody that is endogenous in the subject is an anti-dinitrophenyl (DNP) IgG. In some embodiments, the anti-DNP IgG is present in the subject's serum. In some embodiments, one of PBD1 and PBD2 is a hapten comprising a DNP for binding the anti-DNP IgG.

In some embodiments, the hapten comprises an electron deficient aryl or a carbohydrate. In some embodiments, the electron deficient aryl group is di- or trinitro phenyl. In some embodiments, the carbohydrate comprises digalactose.

In some embodiments, the PBD1 and PBD2 are the same, for example, where dimerization or blockade of dimerization of the same protein is desired.

In some embodiments, one of PBD1 and PBD2 comprise any of the hapten groups described in U.S. Pat. No. 9,296,708. Accordingly, in some embodiments one of PBD1 and PBD2 is a domain that binds to an antibody and is selected from the following groups:
(1) a di- or trinitrophenyl group having the following structure:

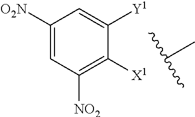

wherein Y¹ is H or NO₂;
X¹ is NR¹, O, CH₂, S(O), SO₂, SO₂O, OSO₂ or OSO₂O; and
R¹ is H, C₁-4alkyl or C(O)C₁₋₄alkyl;
(2) a bicyclic nitro-substituted aromatic group having the following structure:

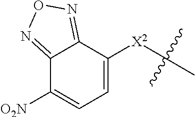

wherein X² is a bond, O, CH₂, NR² or S; and
R² is H, C₁-4alkyl or C(O)C₁₋₄alkyl;
(3) a galactose-containing carbohydrate having the following structure:

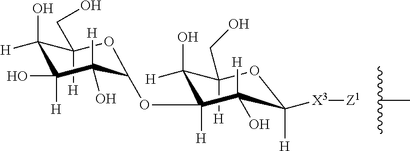

wherein X³ is CH₂, O, NR³ or S;
R³ is H or C₁-4alkyl; and
Z¹ is a bond, monosaccharide, disaccharide, oligosaccharide, glycoprotein or glycolipid; and
(4) a group having the following structure:

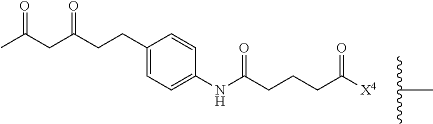

wherein X⁴ is O, CH₂ or NR⁴; and
R⁴ is H, C₁-4alkyl or C(O)C₁₋₄alkyl.

In some embodiments, X¹ is NR¹ and R¹ is H or C₁₋₃alkyl. In some embodiments Y¹ is H.

In some embodiments, X² is a bond or NR² and R² is H or C₁-3alkyl.

In some embodiments, X³ is O or NR³ and R³ is H or C₁-3alkyl. In some embodiment, Z¹ is a bond. In some embodiments, Z¹ is a monosaccharide or a disaccharide. In some embodiments, the monosaccharide is an aldose such as aldotriose (D-glyceraldehdye, among others), aldotetrose (D-erythrose and D-Threose, among others), aldopentose, (D-ribose, D-arabinose, D-xylose, D-lyxose, among others) or aldohexose (D-allose, D-altrose, D-Glucose, D-Mannose, D-gulose, D-idose, D-galactose and D-Talose, among others). In some embodiment, the monosaccharide is a ketose such as ketotriose (dihydroxyacetone, among others), ketotetrose (D-erythrulose, among others), ketopentose (D-ribulose and D-xylulose, among others) or ketohexose (D-Psicone, D-Fructose, D-Sorbose, D-Tagatose, among others). In some embodiments the monosaccharide is an aminosugar such as galactoseamine, sialic acid, N-acetylglucosamine, among others or a sulfosugar such as sulfoquinovose, among others. In some embodiments Z is a disaccharide such as sucrose (which may have the glucose optionally N-acetylated), lactose (which may have the galactose and/or the glucose optionally N-acetylated), maltose (which may have one or both of the glucose residues optionally N-acetylated), trehalose (which may have one or both of the glucose residues optionally N-acetylated), cellobiose (which may have one or both of the glucose residues optionally N-acetylated), kojibiose (which may have one or both of the glucose residues optionally N-acetylated), nigerose (which may have one or both of the glucose residues optionally N-acetylated), isomaltose (which may have one or both of the glucose residues optionally N-acetylated), β,β-trehalose (which may have one or both of the glucose residues optionally N-acetylated), sophorose (which may have one or both of the glucose residues optionally N-acetylated), laminaribiose (which may have one or both of the glucose residues optionally N-acetylated), gentiobiose (which may have one or both of the glucose residues optionally N-acetylated), turanose (which may have the glucose residue optionally N-acetylated), maltulose (which may have the glucose residue optionally N-acetylated), palatinose (which may have the glucose residue optionally N-acetylated), gentiobiluose (which may have the glucose residue optionally N-acetylated), mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose or xylobiose, among others. In some embodiments Z¹ is an oligosaccharide such as any sugar of three or more (up to about 100) individual sugar (saccharide) units as described above (i.e., any one or more saccharide units described above, in any order, especially including glucose and/or galactose units as set forth above), or for example, fructo-oligosaccharides, galactooligosaccharides or mannan-oligosaccharides ranging from three to about ten-fifteen sugar units in size. In some embodiments, Z¹ is a glycoprotein such as N-glycosylated or O-glycosylated glycoproteins, including the mucins, collagens, transferring, ceruloplasmin, major histocompatability complex proteins (MHC), enzymes, lectins, selectins, calnexin, calreticulin, or integrin glycoprotein IIb/Ila, among others. In some embodiments Z¹ is a glycolipid such as a glyceroglycolipid (galactolipids or sulfolipids) or a glycosphingolipid, such as cerebrosides, galactocerebrosides, glucocerebrosides (including glucobicaranateoets), gangliosides, globosides, sulfatides, glycophosphphingolipids or glycocalyx, among others.

In some embodiments, Z¹ is a bond or a glucose or glucosamine (such as N-acetylglucosamine). In some embodiments, Z¹ is linked to a galactose residue through a hydroxyl group or an amine group on the galactose of Gal-Gal, suitably a hydroxyl group.

In some embodiments, X⁴ is NR⁴ and R⁴ is H or C₁-3alkyl.

In some embodiments, one of PBD1 and PBD2 is:

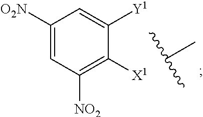

wherein
Y¹ is H or NO₂, suitably H; and
X¹ is NH or O, suitably NH.

In some embodiments, one of PBD1 and PBD2 is independently selected from a variety of monosaccharides or multivalent derivatives thereof recognized by several different serum carbohydrate specific antibodies such as anti-rhamnose and N-acetylglucosamine. In some embodiments, one of PBD1 and PBD2 independently comprise synthetic ligands such as cyclic peptides that bind all serum IgG.

In some embodiments, one of PBD1 and PBD2 comprises Fc receptor (FcR) targeting domains (FTDs), which bind a target protein being a cognate FcR, and the corresponding covalent labeling domain (CLD) comprises a functional group that, on binding of the FTD to the cognate FcR, forms a covalent bond with a nucleophilic group in the FcR. In such embodiments, the other of PBD1 and PBD2 comprises a moiety that binds a protein that is the intended target of the immune cell.

In some embodiments, the FcR targeting domain (FTD) is a synthetic molecule comprising a binding domain which binds to a cognate FcR, on an immune cell. Any suitable FTD may be used depending on the specific FcR being targeted. In some embodiments, the FTD binds FcγR, optionally selected from CD64, CD32, CD16a, and CD16b. In some embodiments, the FcR is CD64. In some embodiments, the FTD comprises circular peptide 33 (CP33) having the sequence VNSCLLLPNLLGCGDD (SEQ ID NO: 1), wherein C4 and C13 form a disulfide bond, or a functional variant thereof. In some embodiments, the FTD comprises a circular peptide 33 (CP33) having the sequence VNSCLLLPNLLGCDGD (SEQ ID NO: 2), wherein C4 and C13 form a disulfide bond, or a functional variant thereof. In some embodiments, the FTD comprises a CP33 having the sequence of SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof. In some embodiments, CP33 comprises a peptide having the sequence of SEQ ID NO: 3, wherein X is K and wherein C5 and C14 form a disulfide bond. In some embodiments, CP33 comprises a peptide having the sequence of SEQ ID NO: 4, wherein X is K and wherein C5 and C14 form a disulfide bond. In some embodiments, CP33 comprises a peptide having the sequence of SEQ ID NO: 7, wherein X is K and wherein C7 and C16 form a disulfide bond. In some embodiments, CP33 comprises a peptide having the sequence of SEQ ID NO: 8, wherein X is K and wherein C7 and C16 form a disulfide bond. In some embodiments, CP33 comprises a peptide having the sequence of SEQ ID NO: 9, wherein X is K and wherein C5 and C14 form a disulfide bond. In some embodiments, CP33 comprises a peptide having the sequence of SEQ ID NO: 10, wherein X is K and wherein C5 and C14 form a disulfide bond. In some embodiments, CP33 comprises a peptide having the sequence of SEQ ID NO: 11, wherein X¹ is K, wherein X¹⁸ is K, and wherein C5 and C14 form a disulfide bond. In some embodiments, CP33 comprises a peptide having the sequence of SEQ ID NO: 12, wherein X¹ is K, wherein X¹⁸ is K, and wherein C5 and C14 form a disulfide bond. In some embodiments, the CP33 peptide is C-terminally amidated. In some embodiments, the CP33 peptide is not C-terminally amidated.

In some embodiments, the PBD comprises other tumor antigen binding ligands such as, without limitation, synthetic peptides which bind uPAR (e.g. as described in Ploug M, et al., Peptide-derived antagonists of the urokinase receptor, affinity maturation by combinatorial chemistry, identification of functional epitopes, and inhibitory effect on cancer cell intravasation. Biochemistry. 2001 Oct. 9; 40 (40): 12157-68, incorporated herein by reference, or functional variants thereof). In an embodiment, the synthetic peptide which binds uPAR comprises an amino acid sequence of L-Lys-Gly-Gly-L-Ser-Gly-L-Asp-L-Cha-L-Phe-D-Ser-D-Arg-L-Tyr-L-Leu-L-Trp-L-Ser (SEQ ID NO: 13), or a functional variant thereof. In an embodiment, the synthetic peptide which binds uPAR comprises an amino acid sequence of L-Lys-Gly-Gly-L-Ser-Gly-L-Asp-L-Cha-L-Phe-D-Ser-D-Arg-L-Ala-L-Leu-L-Trp-L-Ser (SEQ ID NO: 14) as used herein, or functional variant thereof. In some embodiments, the PBD comprises other tumor antigen binding ligands such as, without limitation, HER2, folate receptor binding molecules such as folate or methotrexate, Toll-like receptor (TLR) agonists, or PD-1/PD-L1 antagonists. The PBD may also include Integrin binding ligands such as the Knottin peptide and other RDG mimetics. The PBD may also include therapeutic antibodies, scFv, aptamers, and other biologics.

### Covalent Labeling Domains (CLD)

Covalent Labeling Domains (CLD), including CLD1 and CLD2, are labelling domains comprising a functional group that forms an irreversible covalent bond with nucleophile in the target protein that is proximal to the corresponding PBD binding site. By “proximal” it is meant that the nucleophile is located in an area that, when the compound of the application is bound to the target protein via one of the PBDs, the nucleophile is in a spatial location to react with the CLD. For example, in some embodiments, the distance between the nucleophile and the CLD is about 2 Å to about 10 Å. In some embodiments, CLD1 and CLD2 each independently comprise an electrophilic functional group that reacts with a nucleophilic moiety of an amino acid in the first and second target proteins, respectively. In some embodiments, the amino acid nucleophile is an amine (NH₂) or a thiol (SH).

In some embodiments, CLD1 and CLD2 each independently comprise a sulfur fluoride exchange (SuFEx) functional group, a N-acyl N-alkyl sulfonamide (NASA) functional group, a sulphur triazole exchange (SuTEX) functional group, an acrylamide functional group, an acetamide functional group, an imino-boronate functional group, an imine-forming electrophilic functional group, a p-nitrophenyl ester functional group, a 1,3,5-triazines functional group, an o-nitrobenzoxadiazole functional group, or an imidazole functional group.

In some embodiments, the imidazole group has the following structure:

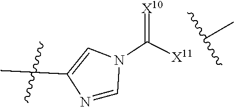

wherein
X¹⁰ is S, O or NR⁶;
X¹¹ is O or NR⁷; and
R⁶ and R⁷ are independently H or C1-4alkyl.

In some embodiments, X¹⁰ and X¹¹ are both O.

A person skilled in the art would appreciate that there are many other functional groups that may be used in CLD1 and/or CLD2. Such group would be compatible with a biological environment and would react with a nucleophile to form a covalent bond.

In some embodiments, CLD1 and CLD2 each independently comprise a fluorosulfate, fluorosulfonate or sulfonyl fluoride group. In some embodiments, CLD1 and CLD2 each independently comprise an aryl sulfonyl fluoride group. In some embodiments, the aryl sulfonyl fluoride group has the following structure:

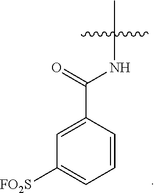

In some embodiments, aryl sulfonyl fluoride electrophiles, as sulfur fluoride exchange (SuFEx) functional groups are advantageous as they undergo significant increases in proximal labeling-effective molarity, react with diverse amino acids proximal to the binding site, and can be efficiently incorporated into complex CGM molecular formats including peptides or carbohydrates.

In some embodiments, CLD1 and/or CLD2 incorporate click chemistry handles to enable for a subsequent 2-step ligation of the corresponding PBD to the acceptor on the target protein using “click” chemistry processes. Click chemistry is used in the art to describe a class of “bio-orthogonal” reactions which are often used for attaching a probe or substrate of interest to a specific biomolecule in a process called bioconjugation which can take place in vitro or directly in vivo. This class of biocompatible small molecule reactions may include, for example, [3+2] cycloadditions such as the Huisgen 1,3-dipolar cycloaddition, thiol-ene reactions, Diels-Alder reactions and inverse electron demand Diels-Alder reactions, [4+1] cycloadditions between isonitriles (isocyanides) and tetrazines. In some embodiments, the click chemistry is a copper catalyzed reaction of an azide and an alkyne to form a triazole.

Other functional groups may be used in CLD1 and/or CLD2. Such group would be compatible with a biological environment and would react with a nucleophile in the target protein to form a covalent bond.

### Linker Groups

A person of skill in the art would appreciate that the linkers L1, L2 and L3 should have a length and spatial orientation appropriate to link the PBD moieties together and/or through a CLD, and the CLD moieties with the remainder of the molecule. In some embodiments, a linker is also incorporated to provide appropriate distance and/or spatial orientation of a protein binding domain and a covalent labeling domain towards a target protein. In some embodiments, the linker rigidity and length is tuned to maximize labeling kinetics and further comprises rigidifying elements such as carbocycles, heterocycles, aromatics and/or heteroaromatics.

Linkers are any molecular structure that joins two or more other molecular structures together and that are compatible with a biological environment. In some embodiments, the linker moiety comprises at least one functional group selected from an ester, amide, ether, thioether, thioamide, thioester and amine.

In some embodiments, L1, L2 and L3, are independently a direct bond, C₁₋₂₀ alkylene, optionally interrupted by triazolyl, piperidinyl, pyrrolidinyl, and/or one or more heteromoieties such as O, S, S(O), SO₂, OSO₂, SO₂O, OSO₂O, NR⁸, C(O), NHC(O), or C(O)NH, wherein R⁸ is H or C1-4alkyl. In some embodiments, L1, L2 and L3 each independently comprises a group having the following structure:

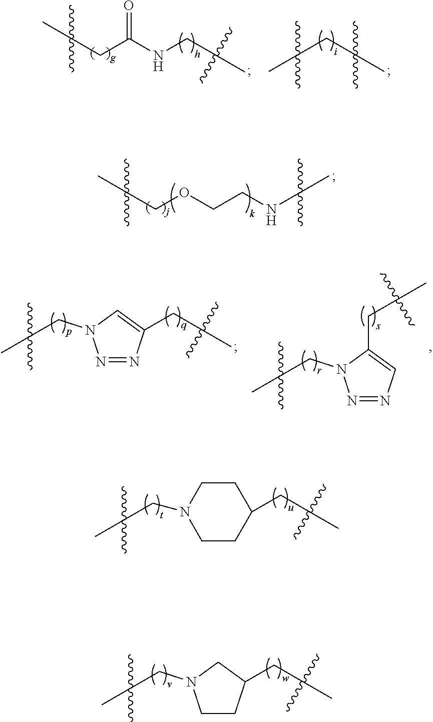

or any combination thereof,
wherein, g, h, i, j, k, p, q, r, s, t, u, v and w are, independently, 1, 2, 3, 4, 5, 6, 7, 8, 9 or 10.

In some embodiments, j is 2 and k is 3. In some embodiments, g is 1 and h is 2. In some embodiments, i is 1, 2, 3, 4, 5, 6, 7, 8, 9 or 10. In some embodiments, p, q, r and s are, independently, 1, 2, 3 or 4.

In some embodiments, L1, L2 and L3, are independently a direct bond,

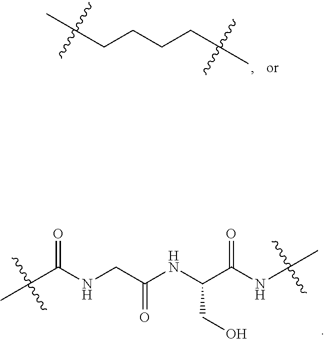 [SMILES: *CCCC* ; *NC(=O)[C@H](CO)NC(=O)CNC(*)=O]

In some embodiments, the present application includes a compound that is:

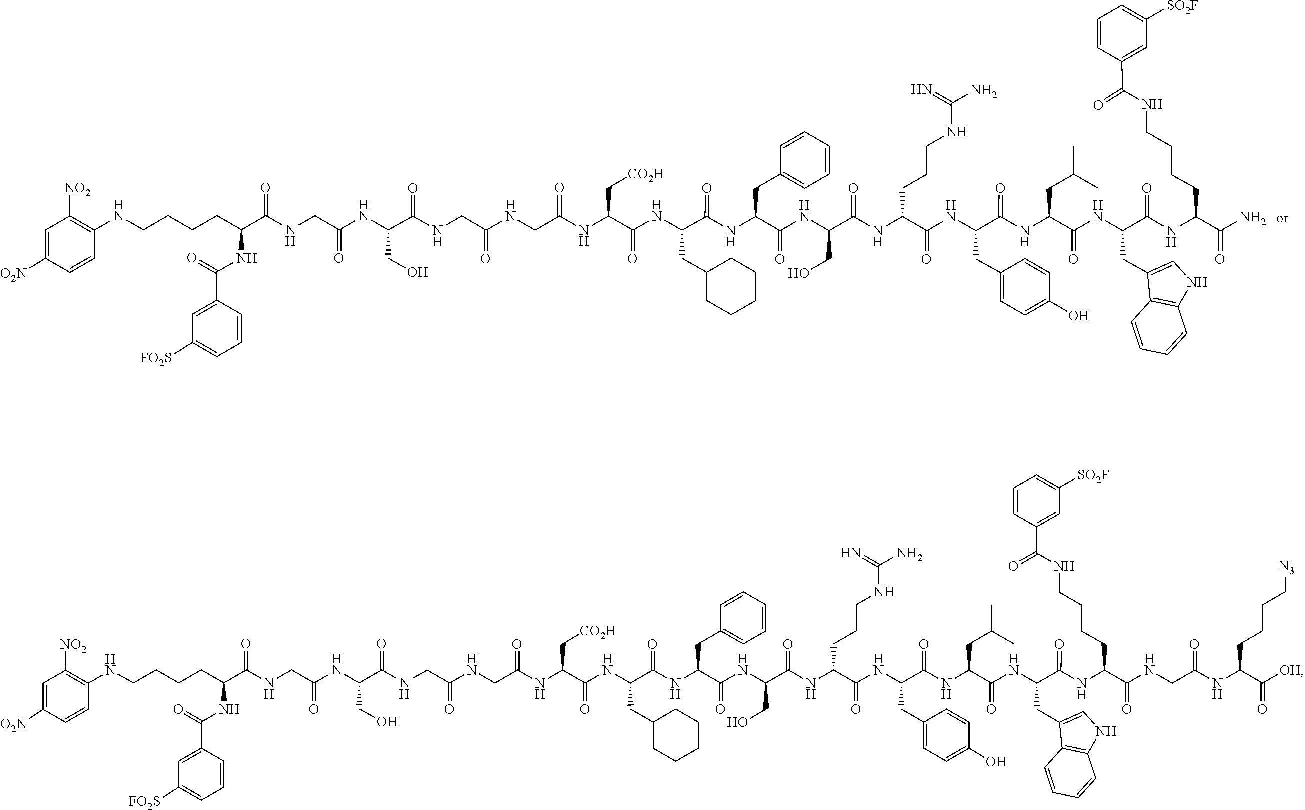

or a pharmaceutically acceptable salt and/or solvate thereof.

In some embodiments, the pharmaceutically acceptable salt is an acid addition salt or a base addition salt. In some embodiments, for pharmaceutical methods and uses on human or animal subjects, the salt is a pharmaceutically acceptable salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66 (1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application. The compounds of the application also include those in which one or more radioactive atoms are incorporated within their structure.

The compounds of the present application may also comprise alternate isotopes of the atoms comprised therein. For example, one or more of available hydrogen atoms are independently replaced with deuterium.

### Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the subject or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

In the above, the term “a compound” also includes embodiments wherein one or more compounds are referenced.

### Target Proteins

In some embodiments, at least one of the first and second target proteins is an immune cell receptor. In some embodiments, the immune cell is selected from monocytes, macrophages, polymorphonuclear cells, erythrocytes, megakaryocytes, neutrophils, basophils, eosinophils, dendritic cells, natural killer (NK) cells B cells, lymphocytes and T cells. In some embodiments, the immune cell is a T cell. In some embodiments, the immune cell is an engineered T cell. In some embodiments, the engineered T cell comprises a synthetic antigen receptor (SAR).

In some embodiments, the first target protein is an antibody and the second target protein is a cell surface receptor. In some embodiments, the first target protein is an antibody and the second target protein is a tumor antigen. In some embodiments, PBD1 comprises a hapten, including, without limitation, the haptens disclosed herein, that selectively binds to an antibody comprising a hapten binding site and PBD2 comprises a cell surface receptor binding domain. In some embodiments, PBD1 comprises a hapten that selectively binds to an antibody comprising a hapten binding site and PBD2 comprises a tumor antigen binding domain.

In some embodiments, the first and second target proteins are each a cell surface receptor. In some embodiments, PBD1 selectively binds to an immune cell receptor and PBD2 selectively binds to a cell surface receptor. In some embodiments, PBD1 selectively binds to an immune cell receptor and PBD2 selectively binds to a tumor antigen.

In some embodiments, the first and second target proteins are each independently selected from an urokinase plasminogen activating receptor (uPAR), a prostate-specific membrane antigen (PSMA), a human epidermal growth factor receptor 2 (HER2), an integrin, CD38, programmed death-ligand-1 (PD-L1), a G protein-coupled receptor (GPCR), a Kirsten rat sarcoma virus (KRAS), a vascular endothelial growth factor (VEGF) and a folate receptor.

### III. Methods and Uses of the Application

The compounds of the application, herein defined as bicovalent protein binding molecules, have been shown to covalently bind to the target proteins and thus irreversibly link the target proteins and, in some embodiments, target cells.

Accordingly, in some embodiments, the present application includes a method for forming ternary protein complexes, either in a biological sample or in a subject, comprising administering an effective amount of a compound or composition of the application to the biological sample or subject. Also provided is use of a compound or composition of the application for forming ternary protein complexes in a biological sample or in a subject. Further provided is a compound or composition of the application for use in forming ternary protein complexes in a biological sample or in a subject.

In some embodiments, administering or using the compound or composition of the application comprises first exposing the compound or composition to the first target protein ex vivo, so that the PBD1 binds its target protein and a covalent bond between the target protein and the compound of the application is formed and then administering the resulting complex to the subject, whereby the resulting complex then binds via the PBD2 to its target in the biological sample or subject. In some embodiments, the use of the compound or composition of the application comprises use of the compound or composition covalently bound to the first target protein via the PBD1, whereby upon use the resulting complex then binds via the PBD2 to its target in the biological sample or subject.

A person skilled in the art can readily choose the first and second PBD depending on the intended benefit of bringing the two proteins in proximity of each other. As described herein, target proteins and their PBDs are known and the selection of suitable PBDs for a particular therapeutic use can be made by a person skilled in the art.

Accordingly, also included is a method for recruiting an antibody or an immune cell for immunotherapy, either in a biological sample or in a subject, comprising administering an effective amount of a compound or composition of the application to the biological sample or subject, wherein the compound or composition comprises PBD1, which selectively binds to the antibody or immune cell and PBD2, which selectively binds to the target, such as an antigen on a cancer cell. Also included is a use of a compound or composition of the application for recruiting an antibody or an immune cell for immunotherapy in a biological sample or subject in need thereof. Further provided is a use of a compound or composition of the application in the manufacture of a medicament for immunotherapy in a biological sample or subject in need thereof. Even further provided is a compound or composition of the application in the for use in immunotherapy in a biological sample or subject in need thereof.

Further included is a method for recruiting an antibody or an immune cell and targeting a cell for provoking an immune response to the cell, either in a biological sample or in a subject, comprising administering an effective amount of a compound or composition of the application to the biological sample or the subject, wherein PBD1 selectively binds the antibody or a protein on the surface of the immune cell and PBD2 selectively binds a protein on the surface of the cell. Further provided is use of a compound or composition of the application for provoking an immune response to a cell in a biological sample or subject in need thereof, wherein PBD1 selectively binds the antibody or a protein on the surface of the immune cell and PBD2 selectively binds a protein on the surface of the cell. Also provided is use of a compound or composition of the application in the manufacture of a medicament for provoking an immune response to a cell in a biological sample or subject in need thereof, wherein PBD1 selectively binds the antibody or a protein on the surface of the immune cell and PBD2 selectively binds a protein on the surface of the cell. Even further provided is a compound or composition of the application for use in provoking an immune response to a cell in a biological sample or subject in need thereof, wherein PBD1 selectively binds the antibody or a protein on the surface of the immune cell and PBD2 selectively binds a protein on the surface of the cell. In some embodiments, the immune response comprises targeted killing of the cell or phagocytosis of the target cell. In some embodiments, the protein on the surface of the cell comprises a tumor antigen on the surface of a cancer cell and the immune response comprises targeted killing of the cancer cell.

The present application also includes a method of treating a disease, disorder, or condition that is treatable by engaging an immune response or by immunotherapy, comprising administering a therapeutically effective amount of a compound or composition of the application to a subject in need thereof, wherein the PBD1 selectively binds to an antibody or immune cell and PBD2 selectively binds to a particular cell, structure organ affected by the disease, disorder or condition. Also provided is use of a compound or composition of the application for treating a disease, disorder or condition that is treatable by engaging an immune response in a subject in need thereof, wherein the PBD1 selectively binds to an antibody or immune cell and PBD2 selectively binds to a particular cell, structure organ affected by the disease, disorder or condition. Further provided is use of a compound or composition of the application in the manufacture of a medicament for treating a disease, disorder or condition that is treatable by engaging an immune response in a subject in need thereof, wherein the PBD1 selectively binds to an antibody or immune cell and PBD2 selectively binds to a particular cell, structure organ affected by the disease, disorder or condition. Even further provided is a compound or composition of the application for use in treating a disease, disorder or condition that is treatable by engaging an immune response in a subject in need thereof, wherein the PBD1 selectively binds to an antibody or immune cell and PBD2 selectively binds to a particular cell, structure organ affected by the disease, disorder or condition. Such diseases, disorders, or conditions that are treatable by immunotherapy or by engaging an immune response include, without limitation, cancer, autoimmune diseases, and allergy, and transplant rejection.

In some embodiments, the compounds of the application bind to and recruit antibodies, such as endogenous antibodies, to the surface of target cells such that the resulting complex comprises the antibody and a protein on the surface of the target cell. These ternary complexes bind activation receptors on immune cells (e.g. CD64 on monocytes, CD3 receptors on T-cells and CD16a receptors on NK cells). The result is the activation of endogenous T cell or NK cell cytotoxicity against the target cell or other immune response provocation. Accordingly, compounds of the application are also effective tools for triggering a cytotoxic response to target cells.

In some embodiments, functionalized cells are generated by contacting immune cells expressing an FcR with a suitable compound of the application, wherein the compound comprises a PBD1 that selectively binds to the FcR, and a PBD2 that selectively binds to a cell of interest to be targeted, wherein the generated functionalized cell is covalently bound to the PBD2. As will be understood, the suitability of a PBD2 will depend on the intended application or use of the functionalized cell. For example, where the intended application of the functionalized cell is for the treatment of a specific cancer, a PBD2 will be one that binds cells of the specific cancer. The skilled person can readily select a suitable PBD2 for the intended application or use.

Accordingly, an aspect described herein includes a method for generating a functionalized cell, the method comprising providing a cell expressing an FcR, and contacting the cell with a compound of the application (comprising a PBD1 that interacts with the FcR under suitable conditions to allow binding of the PBD1 and FcR, and covalent attachment of the covalent labeling domain (CLD) to the FcR, thereby generating a functionalized cell.

Functionalized cells may be generated for example in vitro, ex vivo, or in vivo. For example, an immune cell is contacted in vitro or ex vivo with a compound of the application. Alternatively, the compound of the application is administered to a subject, and the functionalized cell generated in vivo.

The functionalized cells described herein may be directed towards a target cell and exhibit a cytotoxic response to, or phagocytosis of, the target cell. Accordingly, the functionalized cells described herein are effective tools for directing the functionalized cell to a target cell, and/or triggering a cytotoxic response to, or phagocytosis of, the target cell. The target cell is any desired cell. For example, in the context of cancer therapy, the target cell is a cancer cell.

Also described herein is a method for recruiting a functionalized cell described herein to a target cell in a subject, comprising administering an effective amount of a) a compound or composition of the application; or b) a functionalized cell to the subject. Also provided is use of a) a compound or composition of the application; or b) a functionalized cell described herein for recruiting a functionalized cell to a target cell in a subject in need thereof. Further provided is use of a) a compound or composition of the application; or b) a functionalized cell described herein in the manufacture of a medicament for recruiting a functionalized cell to a target cell in a subject in need thereof. Even further provided is a) a compound or composition of the application; or b) a functionalized cell described herein for use in recruiting a functionalized cell to a target cell in a subject in need thereof. In some embodiments, the functionalized cell provokes an immune response to the target cell, such as a cytotoxic or phagocytic response.

Further described herein is a method for targeting and/or recruiting a functionalized cell for provoking an immune response to a target cell in a subject, comprising administering an effective amount of a) a compound or composition of the application, comprising at least one suitable PBD; or b) a functionalized cell comprising at least one suitable PBD to the subject. An aspect also includes use of a) a compound or composition of the application, comprising at least one suitable PBD; or b) a functionalized cell comprising at least one suitable PBD for provoking an immune response to a target cell in a subject. An aspect also includes use of a) a compound of the application, comprising at least one suitable PBD; or b) a functionalized cell comprising at least one suitable PBD in the manufacture of a medicament for provoking an immune response to a target cell in a subject. An aspect also includes a) a compound or composition of the application, comprising at least one suitable PBD; or b) a functionalized cell comprising at least one suitable PBD for use in provoking an immune response to a target cell in a subject.

In some embodiments, the present application includes a method of treating a disease, disorder or condition that is treatable by provoking an immune response, comprising administering a therapeutically effective amount of a compound of the application, to a subject in need thereof.

In some embodiments, the present application includes a use of one or more compounds of the application, for treating a disease, disorder or condition treatable by immunotherapy. The application also includes use of a compound of the application, for the preparation of a medicament for treating of a disease, disorder or condition treatable by immunotherapy.

In some embodiments, the disease, disorder or condition treatable by immunotherapy or by provoking an immune response is cancer, therefore the present application includes a method of treating cancer comprising administering a therapeutically effective amount of a compound or composition of the application to a subject in need thereof. The present application also includes a use of a compound or composition of the application for treatment of cancer as well as a use of a compound of the application for the preparation of a medicament for treatment of cancer. The application further includes a compound or composition of the application for use in treating cancer. In some embodiments, the compound or composition is administered for the prevention of cancer in a subject such as a mammal having a predisposition for cancer.

In some embodiments, the cancer is one that is impacted or treatable by immunotherapy. In some embodiments, the cancer is one that is impacted or treatable by activation of endogenous immune cells. In some embodiments, the cancer is one that is impacted or treatable by provoking an immune response to tumor cells. In some embodiments, the cancer is one that is impacted or treatable by provoking phagocytosis of tumor cells.

In some embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In some embodiments, the cancer is selected from prostate cancer, breast cancer, ovarian cancer and glioblastoma. In some embodiments, the cancer is prostate cancer.

In further embodiments, the present application also includes a method of treating a disease, disorder or condition treatable by immunotherapy, comprising administering to a subject in need thereof a therapeutically effective amount of a compound or composition of the application in combination with another agent useful for treatment of the disease, disorder or condition treatable by immunotherapy. The present application also includes a use of a compound or composition of the application in combination with an agent useful for treatment of a disease, disorder or condition treatable by immunotherapy, for treatment of such disease, disorder or condition.

In some embodiments, the disease, disorder or condition treatable by immunotherapy is cancer and a compound or composition of the application is administered in combination with one or more additional cancer treatments. In some embodiments, the additional cancer treatment is selected from radiotherapy, chemotherapy, targeted therapies such as antibody therapies and small molecule therapies such as tyrosine-kinase and serine-threonine kinase inhibitors, immunotherapy, hormonal therapy and anti-angiogenic therapies.

In some embodiments, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In some embodiments, the compound or composition of the application is administered at least once a week. However, in some embodiments, the compound or composition is administered to the subject from about one time per two weeks, three weeks or one month. In some embodiments, the compound or composition is administered about one time per week to about once daily. In another embodiment, the compound or composition is administered 2, 3, 4, 5 or 6 times daily. The length of the treatment period depends on a variety of factors, such as the severity of the disease, disorder or condition, the age of the subject, the concentration and/or the activity of the compound of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds or compositions are administered to the subject in an amount and for duration sufficient to treat the subject.

In some embodiments, the subject is a mammal. In another embodiment, the subject is human.

A compound or composition of the application is either used alone or in combination with other known agents useful for treating diseases, disorders or conditions as defined above, such as the compounds disclosed herein. When used in combination with other agents useful in treating such diseases, disorders or conditions, it is some embodiments that a compound or composition of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent, and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound of the application is administered initially in a suitable dosage that is adjusted as required, depending on the clinical response. Dosages will generally be selected to maintain a serum level of the compound of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

In the above-described methods and uses, the term “a compound” also includes embodiments wherein one or more compounds are referenced.

### IV. Methods of Preparation

The compounds of the application can be synthesized by any of the techniques that are known to those skilled in the art. For example, some compounds are amendable to standard polypeptide synthetic chemistry techniques, such as a solid-phase Merrifield-type synthesis. A summary of the many such techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

The formation of a desired compound salt is achieved using standard techniques. For example, the neutral compound is treated with an acid or base in a suitable solvent and the formed salt is isolated by filtration, extraction or any other suitable method.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Specific enantiomers or diastereomers of the compounds of the application are available by using corresponding single enantiomers or diastereomers of the corresponding starting materials.

TABLE 1
Compounds of the present application.

| | | | Mass | |
|---|---|---|---|---|
| | | | Spectrometry | Obtained |
| Compound | | | Expected m/z | Molecular |
| Number | Name | Chemical Formula and Structure | (LC-HRMS) | Weight |
| Int-1 | AE105- | C₇₂H₉₈N₁₇O₂₀ | [M + H]⁺ m/z: | [M + H]⁺ m/z: |
| | DNP | | 1521.67 | 1521.15 |
| | | 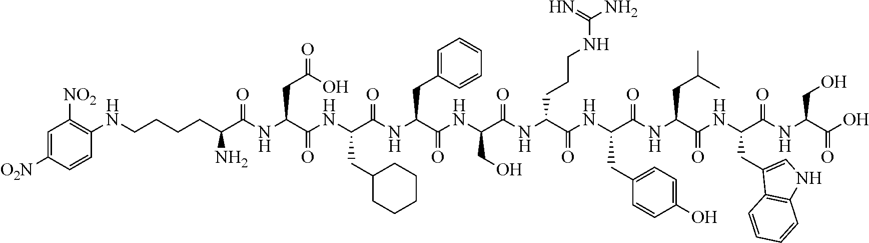 [SMILES: CC(C)C[C@H](NC(=O)[C@H](Cc1ccc(O)cc1)NC(=O)[C@@H](CCCNC(=N)N)NC(=O)[C@@H](CO)NC(=O)[C@H](Cc1ccccc1)NC(=O)[C@H](CC1CCCCC1)NC(=O)[C@H](CC(=O)O)NC(=O)[C@@H](N)CCCCNc1ccc([N+](=O)[O-])cc1[N+](=O)[O-])C(=O)N[C@@H](Cc1c[nH]c2ccccc12)C(=O)N[C@@H](CO)C(=O)O] | | |
| Int-2 | BM | C₈₁H₁₁₃N₂₂O₂₄ | [M + H]⁺ m/z: | [M + H]⁺ m/z: |
| | | | 1778.92 | 1779.33 |
| | | 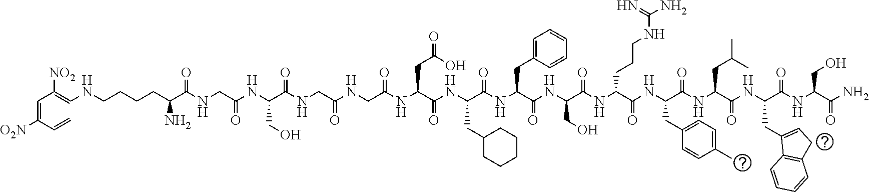 [SMILES: C=C/C(=C\C(=C\NCCCC[C@H](N)C(=O)NCC(=O)N[C@@H](CO)C(=O)NCC(=O)NCC(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](Cc1ccc(C)cc1)C(=O)N[C@@H](CC(C)C)C(=O)N[C@@H](CC1=CCc2ccccc21)C(=O)N[C@@H](CO)C(N)=O)[N+](=O)[O-])[N+](=O)[O-]] | | |
| Int-3 | ^{Ab}cBM | C₃₈H₁₁₆FN₂₂O₂₇S | [M + H]⁺ m/z: | [M + H]⁺ m/z: |
| | | | 1965.07 | 1966.26 |
| | | 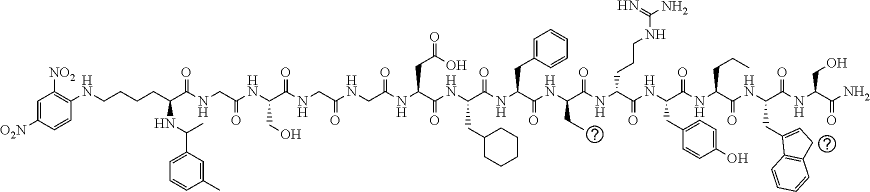 [SMILES: CCC[C@H](NC(=O)[C@H](Cc1ccc(O)cc1)NC(=O)[C@@H](CCCNC(=N)N)NC(=O)[C@@H](CC)NC(=O)[C@H](Cc1ccccc1)NC(=O)[C@H](CC1CCCCC1)NC(=O)[C@H](CC(=O)O)NC(=O)CNC(=O)CNC(=O)[C@H](CO)NC(=O)CNC(=O)[C@H](CCCCNc1ccc([N+](=O)[O-])cc1[N+](=O)[O-])NC(C)c1cccc(C)c1)C(=O)N[C@@H](CC1=CCc2ccccc21)C(=O)N[C@@H](CO)C(N)=O] | | |
| Int-4 | ^{uPAR}cBM | C₉₃H₁₂₅FN₂₃O₂₇S | [M + 2H]²⁺ | [M + 2H]²⁺ |
| | | | m/z: 1024.45 | m/z: 1024.41 |
| | | 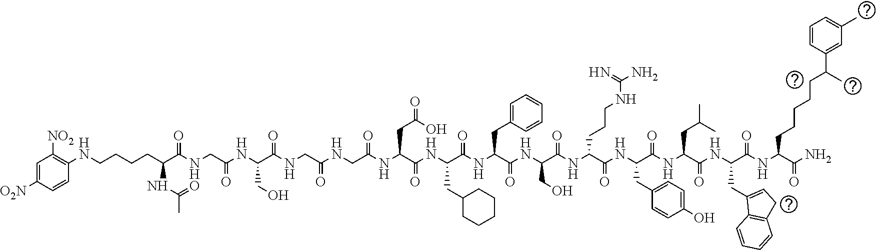 [SMILES: CC(=O)N[C@@H](CCCCNc1ccc([N+](=O)[O-])cc1[N+](=O)[O-])C(=O)NCC(=O)N[C@@H](CO)C(=O)NCC(=O)NCC(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](Cc1ccc(O)cc1)C(=O)N[C@@H](CC(C)C)C(=O)N[C@@H](CC1=CCc2ccccc21)C(=O)N[C@@H](CCCCCC(C)c1cccc(C)c1)C(N)=O] | | |
| Int-5 | ^{uPAR}cBM-2 | C₈₇H₁₂₁FN₂₃O₂₇S | [M + 2H]²⁺ | [M + 2H]²⁺ |
| | | | m/z: 986.43 | m/z: 986.37 |
| | | 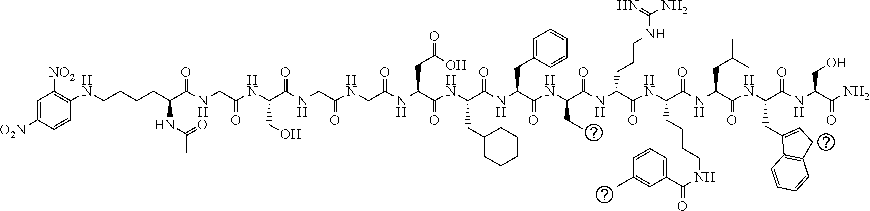 [SMILES: CC[C@@H](NC(=O)[C@H](Cc1ccccc1)NC(=O)[C@H](CC1CCCCC1)NC(=O)[C@H](CC(=O)O)NC(=O)CNC(=O)CNC(=O)[C@H](CO)NC(=O)CNC(=O)[C@H](CCCCNc1ccc([N+](=O)[O-])cc1[N+](=O)[O-])NC(C)=O)C(=O)N[C@H](CCCNC(=N)N)C(=O)N[C@@H](CCCCNC(=O)c1cccc(C)c1)C(=O)N[C@@H](CC(C)C)C(=O)N[C@@H](CC1=CCc2ccccc21)C(=O)N[C@@H](CO)C(N)=O] | | |
| Int-6 | ^{uPAR}cBM-3 | C₉₀H₁₁₈FN₂₀O₂₈S | [M + 2H]²⁺ | [M + 2H]²⁺ |
| | | | m/z: 989.91 | m/z: 989.83 |
| | | 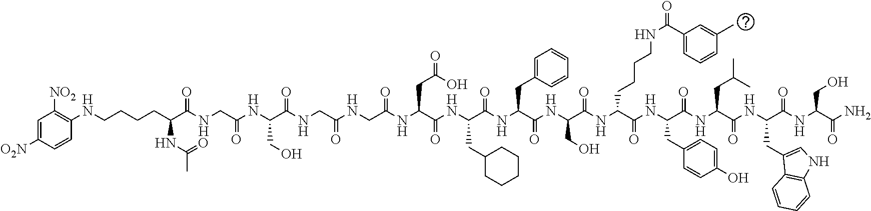 [SMILES: CC(=O)N[C@@H](CCCCNc1ccc([N+](=O)[O-])cc1[N+](=O)[O-])C(=O)NCC(=O)N[C@@H](CO)C(=O)NCC(=O)NCC(=O)N[C@@H](CC(=O)O)C(=O)N[C@@H](CC1CCCCC1)C(=O)N[C@@H](Cc1ccccc1)C(=O)N[C@H](CO)C(=O)N[C@H](CCCCNC(=O)c1cccc(C)c1)C(=O)N[C@@H](Cc1ccc(O)cc1)C(=O)N[C@@H](CC(C)C)C(=O)N[C@@H](Cc1c[nH]c2ccccc12)C(=O)N[C@@H](CO)C(N)=O] | | |
| Int-7 | ^{uPAR}cBM-4 | C₉₂H₁₂₅FN₂₃O₂₆S | [M + 2H]²⁺ | [M + 2H]²⁺ |
| | | | m/z: 1010.45 | m/z: 1010.44 |
| | | 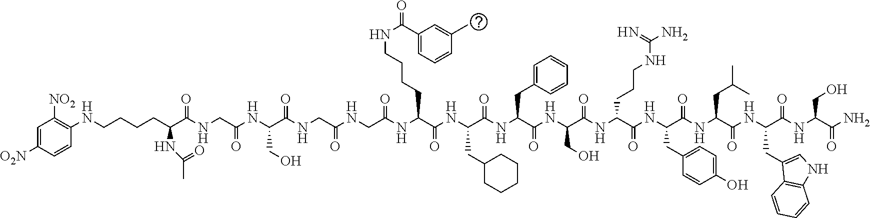 [SMILES: CC(=O)N[C@@H](CCCCNc1ccc([N+](=O)[O-])cc1[N+](=O)[O-])C(=O)NCC(=O)N[C@@H](CO)C(=O)NCC(=O)NCC(=O)N[C@@H](CCCCNC(=O)c1cccc(C)c1)C(=O)N[C@@H](CC1CCCCC1)C(=O)N[C@@H](Cc1ccccc1)C(=O)N[C@H](CO)C(=O)N[C@H](CCCNC(=N)N)C(=O)N[C@@H](Cc1ccc(O)cc1)C(=O)N[C@@H](CC(C)C)C(=O)N[C@@H](Cc1c[nH]c2ccccc12)C(=O)N[C@@H](CO)C(N)=O] | | |
| I-1 | CGM | C₉₈H₁₂₅F₂N₂₃O₂₉S₂ | [M + 2H]²⁺ | [M + 2H]²⁺ |
| | | | m/z: 1095.92 | m/z: 1096.06 |
| | | 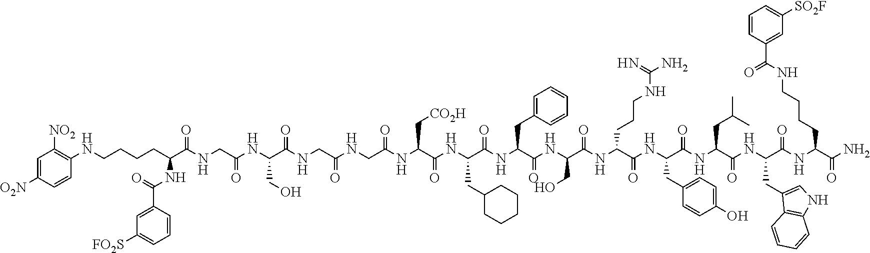 | | |
| I-2 | CGM- | C₁₀₆H₁₃₈F₂N₂₇O₃₂S₂ | [M + 2H]²⁺ | [M + 2H]²⁺ |
| | Azide | | m/z: 1202.48 | m/z: 1201.69 |
| | | 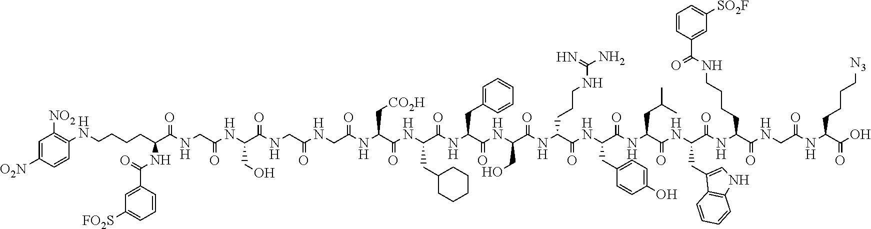 | | |

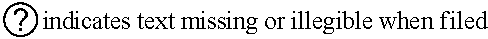

### Examples

The following non-limiting examples are illustrative of the present application:

### Results

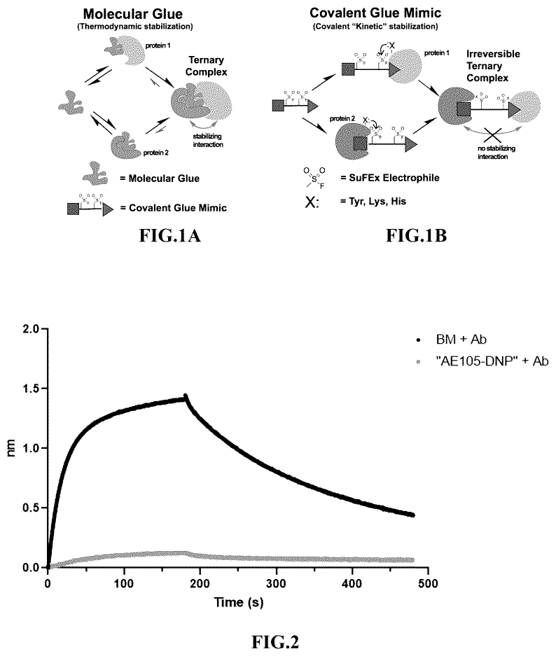

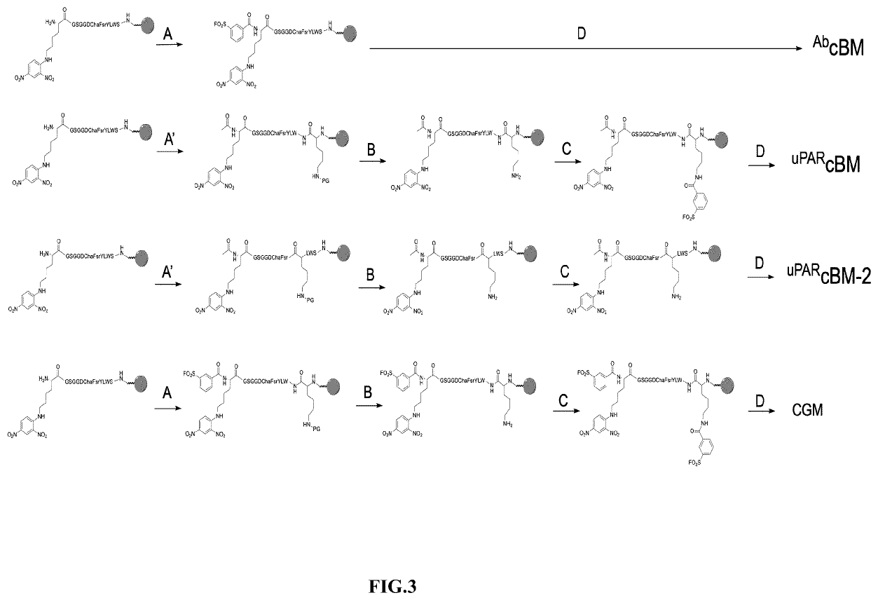

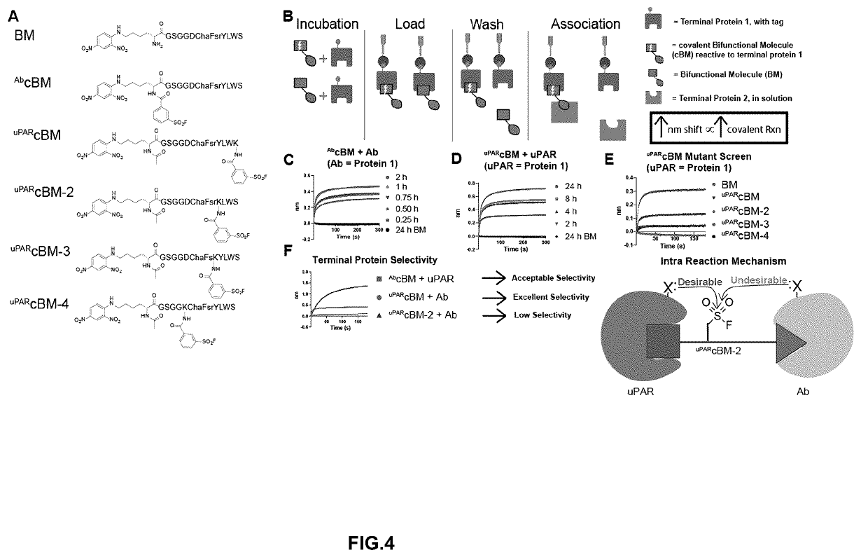

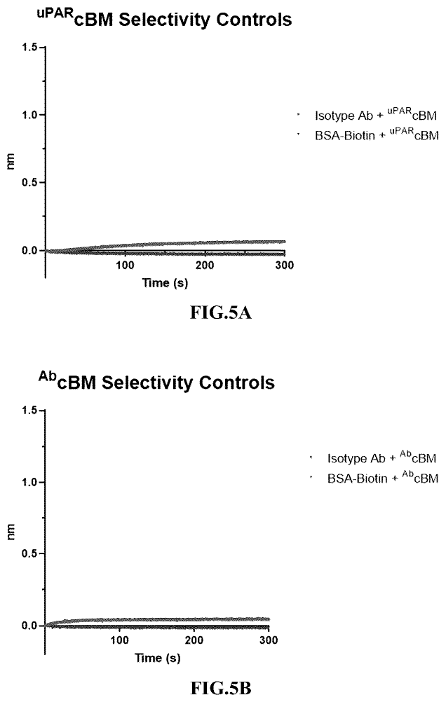

Evaluation of bifunctional molecules that covalently engage either ternary complex protein. Mono-covalent bifunctional molecules (cBM) were chemically synthesized to covalently engage anti-DNP IgG antibody or urokinase plasminogen activating receptor (uPAR) in a ternary complex. uPAR represents a well-established target widely expressed on a variety of cancer types. uPAR is also involved in several tumor growth processes including metastasis via binding to its endogenous high affinity ligand, the urokinase plasminogen activator (uPa.) 19 Non-covalent control bifunctional molecules (BM) were also synthesized to discern covalent versus non-covalently stabilized ternary complexes. To engage uPAR, BMs/cBMs were equipped with a uPAR binding peptide derived from the literature, including “AE133”. “AE105” and “AE137” peptides.²⁰ This was appended to a DNP ligand to engage anti DNP IgG through an optimized linker that was established to be important to mediate ternary complex formation (FIG. 2). To enable covalent engagement of uPAR or anti-DNP antibody (Ab), a single aryl sulfonyl fluoride “SuFEx” electrophile was incorporated site specifically into BM peptide scaffolds via solid phase synthesis, to generate cBMs (FIG. 3). These two cBMs, for engaging uPAR (^{uPAR}cBM) or the anti-DNP Ab (^{Ab}cBM), each containing a single SuFEx insertion, were first evaluated in a Bio-Layer Interferometry (BLI) ternary complex binding assay (FIG. 4). ^{Ab}cBM tightly pre-organizes the SuFEx close to the DNP binding ligand while ^{uPAR}cBM places the SuFEx on the C terminus of the uPAR binding peptide to maximize its distance from reacting with bound anti-DNP. Identifying cBMs that can selectively label only uPAR (^{uPAR}cBM) or only anti-DNP Ab (^{Ab}cBM) versus off-target proteins would help isolate two positions to insert two SuFEx electrophiles on the bifunctional molecule, furnishing the desired covalent glue mimics (CGMs-compounds I-1 and I-2) that crosslink uPAR with anti-DNP in a ternary complex.

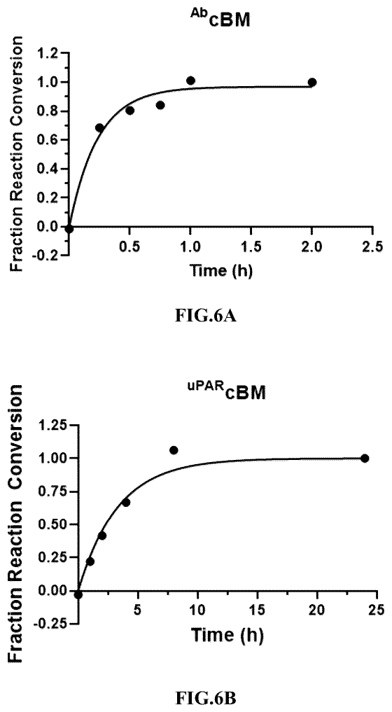

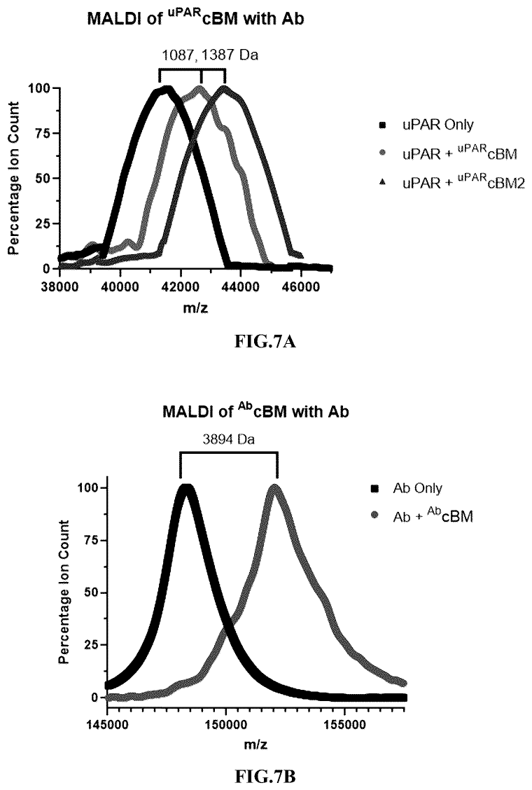

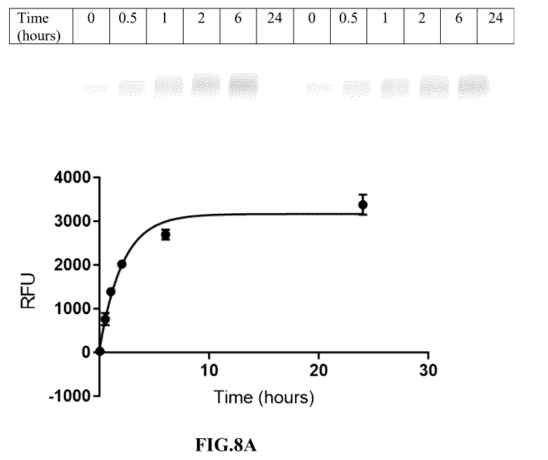

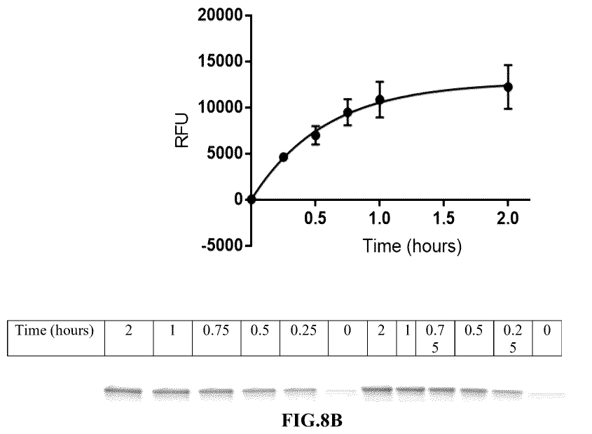

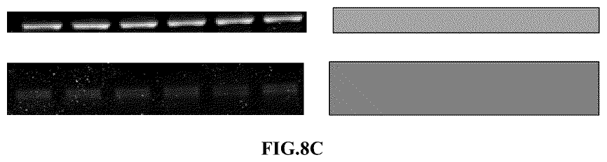

BLI assays begin with an initial incubation step where the compound is incubated with one of the ternary complex proteins (i.e. Protein 1=uPAR or Anti-DNP Ab) containing an affinity tag (FIG. 4, panel B). This complex is then loaded onto a biosensor through the affinity tag, followed by a series of wash steps to remove non-covalent complex. In the final step, the soluble terminal protein is added (Protein 2), which leads to formation of the ternary complex, and a concomitant association signal. Therefore, only ternary complexes stabilized by a covalent linkage generate an association signal. Incubations with either ^{uPAR}cBM or ^{Ab}cBM led to an increase in association signal as a function of increasing incubation times with Protein 1 (i.e. antibody or uPAR) (FIG. 4, panel C and FIG. 4, panel D). In control experiments, baseline association signal was observed when anti-DNP was substituted with non-specific Isotype IgG, or uPAR substituted with BSA, supporting covalent reaction selectivity (FIG. 5A and FIG. 5B). Baseline association signal was observed when non-covalent BM was used (which lacks a SuFEX electrophile), confirming non-covalent complex is dissociated during BLI wash steps. Collectively, these results support the ability of ^{uPAR}cBM and ^{Ab}cBM to form selective covalent complexes with uPAR or anti-DNP, respectively. For ^{Ab}cBM, a fast increase was observed in association plateau with time, which enabled determination of anti-DNP antibody labeling kinetics (k_{obs}~1.2×10⁻³ s⁻¹, FIG. 6A). For ^{uPAR}cBM, a slower increase was observed in association plateau with time, corresponding to a smaller rate constant (k_{obs}~7.5×10⁻⁵ s⁻¹) for labeling uPAR (FIG. 6B). MALDI analysis further confirmed the formation of covalent binary complexes ^{uPAR}cBM-uPAR and ^{Ab}cBM-anti-DNP (FIG. 7A and FIG. 7B). Fluorescent SDS-PAGE assays were also conducted as an orthogonal assay to measure cBM reaction kinetics (FIG. 8A, FIG. 8B and FIG. 8C).

Optimizing the reaction effective molarity of both electrophiles on a single bifunctional molecule enforces formation of the desired covalently cross-linked ternary complex, versus labeling of two off target proteins. Optimizing the reaction effective molarity is also important for enabling electrophile discrimination of/between the two bound ternary complex proteins. This also disfavors two electrophiles reacting with the same bound protein. Since antibody labeling rate was rapid, the aim was to further increase the uPAR labeling rate. ^{uPAR}cBM was designed to minimize competitive antibody labeling at the expense of a slower uPAR labeling rate. Data from a literature uPAR/AE133 derivative co-crystal structure, however, suggested that installation of the electrophile closer to the AE133 N terminus, at the internal tyrosine position, would increase pre-organization with a potential SuFEx reactive tyrosine on uPAR (i.e. Y57) increasing labeling kinetics.²¹ The results from a literature-reported AE133 alanine scan suggested that this internal AE133 tyrosine and two additional residues (d-Arg, Asp) could be substituted with a SuFEx functionalized amino acid without perturbing uPAR binding. With this information, additional analogs ^{uPAR}cBM (2-4) were synthesized, with SuFEx substituted at one of these three locations, for evaluation in further BLI assays (FIG. 4, panel E). In these assays, it was observed that SuFEx positioning dramatically modulates the rate of uPAR labeling. ^{uPAR}cBM-2, predicted to maximize the proximity of SuFEx to Y57 on uPAR demonstrated the most rapid uPAR labeling.

Next, the design strategy was evaluated for the capacity to covalently discriminate each protein within a ternary complex. A lack of proper discrimination or “intra selectivity” can occur because the binding of one protein pre-organizes both electrophiles to react before binding to Protein 2. This represents a unique and significant challenge compared to conventional covalent inhibitor development which labels a single protein. To test this, the assays described above were repeated, however the cBM was incubated with the opposite terminal protein (i.e. ^{uPAR}cBM was incubated with anti-DNP Ab to bind ProG probes, and ^{Ab}cBM was incubated with biotinylated uPAR to bind streptavidin probes) (FIG. 4, panel F). In this assay, proximity labeling of the undesired ternary complex terminal protein will lead to increases in association signal. Here, it was observed that protein incubations with ^{Ab}cBM gave rise to low association signal amplitudes, consistent with low uPAR labeling from within the non-covalent binary complex. This suggests ^{Ab}cBM will demonstrate intra selectivity for bound antibody versus uPAR within a ternary complex. ^{Ab}cBM binding to the deep cleft on uPAR positions the electrophile closer to the uPAR surface, explaining the observed low degree of uPAR labeling. Protein incubations with ^{uPAR}cBM gave rise to baseline association signal amplitude, indicating a lack of labeling from within the non-covalent binary complex. This suggests ^{uPAR}cBM will demonstrate high intra selectivity for bound uPAR versus antibody within a ternary complex. Protein incubations with ^{uPAR}cBM-2, however, indicated efficient albeit undesirable anti-DNP labeling from within the non-covalent binary complex. This can be rationalized by the closer positioning of the SuFEx to the DNP ligand compared to ^{uPAR}cBM. Despite the faster uPAR labeling kinetics potentially achievable, a covalent glue mimic (CGM) based on ^{uPAR}cBM-2 could also label anti-DNP via the electrophile intended for uPAR, and fail to crosslink the ternary complex. For this reason, the CGM was designed to incorporate the two SuFEx electrophiles at the combined positions used in both ^{uPAR}cBM and ^{Ab}cBM.

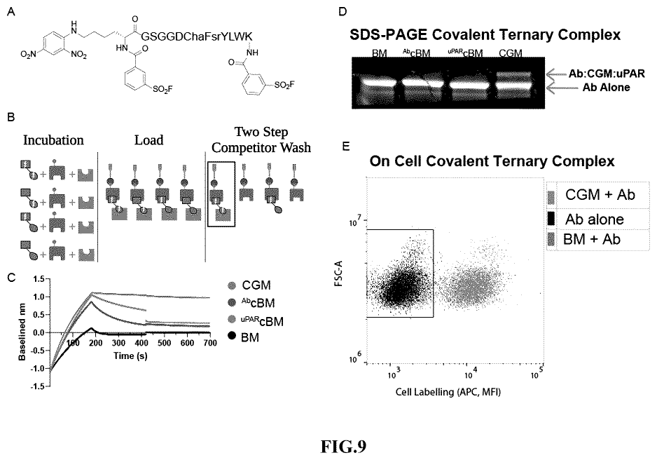

Ternary complex covalent crosslinking using a Covalent Glue Mimic (CGM). A CGM was synthesized and tested using a modified BLI Assay (FIG. 9, panel A). In this assay both biotinylated uPAR, and unmodified anti-DNP, were incubated together with one of CGM, ^{uPAR}cBM, ^{Ab}cBM, or BM, before loading onto the biosensor. Successful ternary complex formation gives rise to an association signal. Next, the loaded probe is submerged in dissociation “wash” solutions containing uPAR competitor ligand followed by anti-DNP competitor ligand to remove non-covalent binding complexes (FIG. 9, panel B). In this experiment, it was observed that each covalent compound gave rise to similar association plateaus, and that these plateaus were significantly higher than that achieved by non-covalent BM (FIG. 9, panel C). The comparable association signals between covalent compounds support their ability to form comparable amounts of ternary complexes, and with increased kinetic stability compared to BM. Ternary complex signal associated with the ^{Ab}cBM condition quickly decreased during the first wash step which contains free uPAR competitor ligand, consistent with rapid dissociation of ^{Ab}cBM from immobilized uPAR. This supports the ability of ^{Ab}cBM to covalently discriminate between uPAR and anti-DNP in a ternary complex, and only label anti-DNP. Ternary complex signal associated with the ^{uPAR}cBM condition decreased more slowly during the first wash step compared to ^{Ab}cBM, and rapidly during the second wash step. This observation is consistent with ^{uPAR}cBM covalently linked to uPAR on the probe. The slow dissociation observed during the first wash step is attributed to slow dissociation of anti-DNP antibody in the absence of DNP competitor. In the second wash step which contains DNP competitor, the remaining non-covalently bound anti-DNP rapidly dissociates from ^{uPAR}cBM “linked” to uPAR coated probe. This result also supports the ability of ^{uPAR}cBM to covalently discriminate between uPAR and anti-DNP in a ternary complex and only label uPAR. Ternary complex signal associated with the CGM condition, however, remained unchanged in the presence of both DNP and uPAR competitor wash steps. This is consistent with complete conversion to covalently cross-linked ternary complex (FIG. 9, panel C).

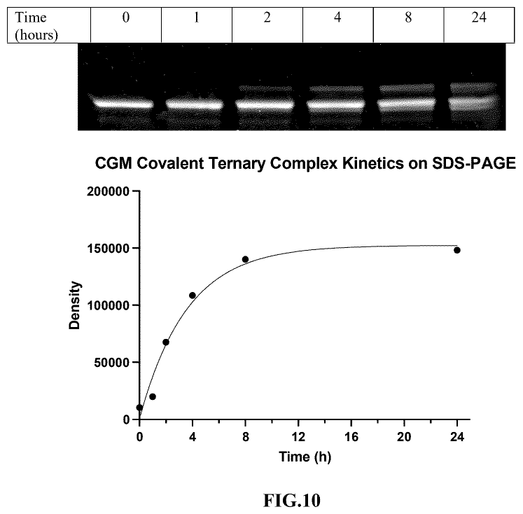

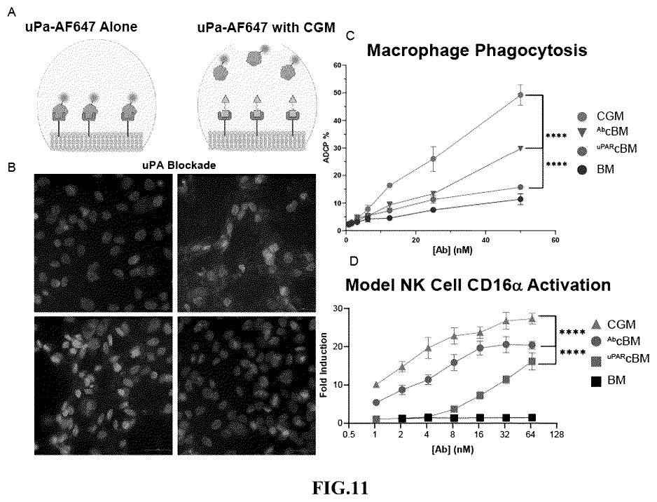

As an orthogonal method to confirm the formation of covalently crosslinked ternary complex, the reaction was monitored by SDS-PAGE. The incubation of uPAR, anti-DNP and CGM led to the formation of a new larger molecular weight band corresponding to both ternary complex proteins, that is not observed using mono-covalent analogs (FIG. 9, panel D). Quantification of increased band intensity with incubation time enabled estimation of covalent crosslinking kinetics (FIG. 10) which appear to be similar to and likely limited by the uPAR labeling step as anticipated.

Next, the CGM ability to covalently crosslink ternary complexes directly on the surface of tumor cells was evaluated. Here anti-DNP-AF647 conjugates were incubated with both CGM and uPAR+A172 cells. The cells were washed to remove non-covalent complex, and their fluorescence measured by flow cytometry. It was observed that BM conditions gave rise to baseline cell associated fluorescence supporting the absence of ternary complexes. The CGM condition however led to a significant fluorescence increase that was resistant to wash steps, supporting covalently crosslinked ternary complex formation on the tumor cell surface (FIG. 9, panel E). Collectively this data supports the first example of a rationally designed dual electrophilic bifunctional molecule, that irreversibly stabilizes cell surface ternary complexes comprising non-interacting proteins. Without being bound to theory, this strategy does not rely on endogenous proteins containing a single chemoselective amino acid like cysteine.

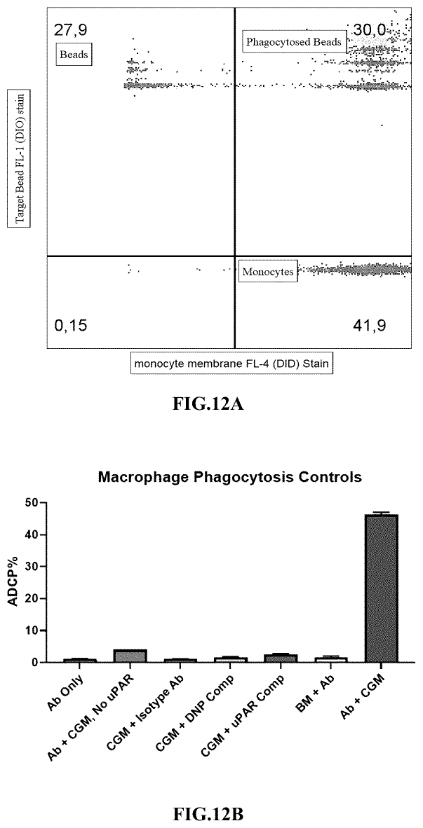

CGM covalent engagement with tumor cell surface uPAR inhibits binding from endogenous competitor ligand urokinase. Proximity-inducing therapeutic applications are susceptible to inhibition by endogenous competitor ligands, especially in the absence of ternary complex positive co-operativity. Ternary complex formation with uPAR is similarly prone to inhibition from urokinase (uPA), a natural high affinity (i.e. sub-nM) ligand for uPAR whose binding initiates tumor proliferation and metastasis. Since CGMs were demonstrated to efficiently react with cell surface uPAR, it was anticipated that they can template protein complexes resistant to uPA competition. This would also contribute an additional mode of potential anti-tumor function independent of immune engagement. To test this, a microscopy assay was developed to image the ability of CGM or BM to block uPa-AF647 conjugate binding to uPAR+A172 glioma brain cancer cells (FIG. 11). In this assay, A172 cells are preincubated with CGM or BM for 15 minutes, followed by co-incubation with uPa-AF647 for one hour. Using a fluorescence widefield microscope, a substantial decrease in cell surface localized fluorescence was observed in the presence of CGM compared to BM. This is consistent with increased blockade of the uPA binding interaction due to covalent stabilization of uPAR complexes with CGM.

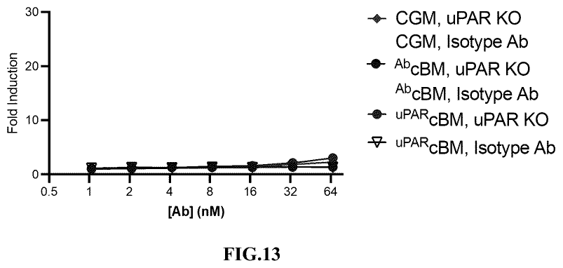

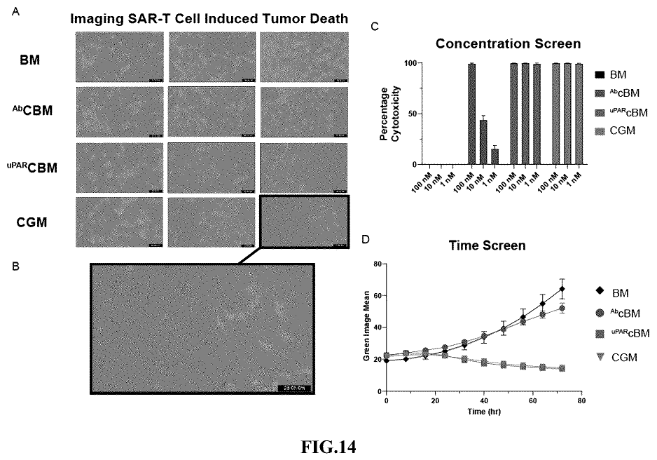

CGM elicits greater efficacy compared to mono-covalent analogs in functional tumor immunotherapy assays. The functional relevance of irreversibly stabilizing ternary complexes was tested in the context of model cell-cell induced proximity. CGM function was studied in an established flow cytometry antibody dependent cellular phagocytosis (ADCP) assay which models macrophage anti-tumor function. In this assay format, uPAR-Biotin, bifunctional molecule, and anti-DNP Ab are incubated overnight. The next day this solution is added to fluorescent streptavidin microspheres to model cancer targets, and fluorescent u937 cultured macrophage immune cells. Successful proximity induction through the formation of tumor antigen-immune complexes leads to target phagocytosis, which can be measured using 2-colour flow cytometry. Here the ability of CGM to promote tumor target phagocytosis was compared directly to ^{Ab}cGM, ^{uPAR}cGM, and BM. All compounds were able to selectively mediate ADCP, however, a significant potency and efficacy advantage using the CGM was observed, (FIG. 11, panel C and FIG. 12A, FIG. 12B). These compounds were then evaluated in a Food and Drug Administration (FDA) approved monoclonal antibody CD16a activation assay which models natural killer anti-tumor function via antibody dependent cell mediated cytotoxicity (ADCC). Here, CD16+Jurkat T-cell lines are incubated with uPAR+A172 cells, bifunctional compound and antibody. Jurkat T cells are engineered to couple antibody-dependent CD16a activation to luciferase expression, resulting in a detectable “fold induction” signal increase. CGMs were observed to selectively induce CD16 immune activation with a significant enhancement in both potency and efficacy compared to the other analogs (FIG. 11, panel D and FIG. 13). Without being bound to theory, no dose of BM or monocovalent analogs could match the efficacy achieved via CGM, despite the likelihood that the number of ternary complexes formed are comparable. This supports previous reports on the importance of kinetically stabilizing tumor-immune receptor complexes to enforce receptor activation signalling.^{9,22}

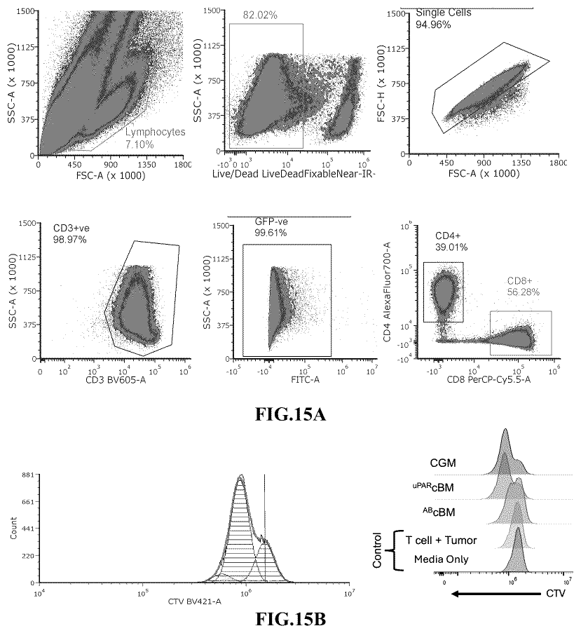

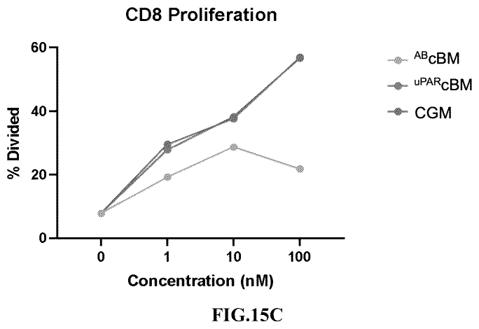

Covalent stabilization of tumor antigen binding within cell-cell ternary complexes induces T cell-tumor cell proximity, leading to tumor eradication. In addition to antibody-based proximity inducing strategies, universal T cell approaches have emerged as a promising cellular tumor immunotherapeutic strategy. Here the T cell is engineered with a receptor that recognizes a bifunctional molecule, which in turn templates a ternary complex with tumor antigens to activate tumoricidal T cell function.²⁴-26 Using a recently described universal T cell system (Serniuck et al., in review) engineered with a synthetic antigen receptor (SAR) that recognizes the DNP ligand, CGM's ability to activate universal T cell tumoricidal function was evaluated. Using live cell imaging, T cell cytotoxic function was measured in response to compound treatment in a co-culture system comprising DNP specific SAR-T cells and GFP+uPAR expressing A172 glioma cells (FIG. 14). A 100% tumor cell cytotoxicity was observed using only 1 nM of CGM or ^{uPAR}cBM in contrast to ^{Ab}cBM and BM. The same trend was observed in T cell proliferation assays where T cell proliferation in response to SAR engagement serves as a measure of T cell activation via cell-cell induced proximity (FIG. 15A, FIG. 15B, and FIG. 15C). Despite high affinity binding via BM and comparable ternary complex formation, covalency appears beneficial for cell-cell induced proximity leading to tumoricidal function. This observation suggests in the context of direct immune cell engagement, covalency to the tumor antigen alone within the ternary complex is sufficient to achieve optimal tumoricidal immune function.

### DISCUSSION

Herein a dual covalent bifunctional molecule strategy to covalently crosslink ternary complexes with tumor immunotherapeutic function is disclosed. The incorporation of two strategic electrophiles within a synthetic chimeric molecule, which can be pre-organized to covalently crosslink two proteins, kinetically “mimicking” molecular glue stabilization, is demonstrated. Notably, molecular glues have been confined to a select few protein: protein interactions. This approach expands the scope to any proximity inducing molecule. Achieving high intra ternary selectivity of the two electrophiles on CBM, while maintaining fast labeling kinetics represents a unique developmental challenge, however, this application demonstrated the ability of CGMs (compounds I-1 and I-2) to efficiently discriminate between two ternary complex proteins.

CGMs rely on reaction kinetic effective molarity for dual covalent crosslinking, making this strategy broadly applicable to irreversibly crosslinking any two endogenous proteins without dependence on cysteines. By tuning the electrophile pre-organization and reaction effective molarity, even modest affinity targeting ligands can be strategically leveraged. It was observed that SuFEx positioning dramatically modulates the rate of uPAR labeling, allowing for significant increases in labelling rate without needing to increase the intrinsic electrophile reactivity. This is advantageous since increasing the latter also favors hydrolysis and off-target labeling.

In immuno-oncology applications of cell-cell induced proximity, it was demonstrated that CGMs can covalently crosslink ternary complexes leading to increased tumoricidal immune function via both macrophage and NK cell receptor activation. It was also demonstrated that covalent engagement of the tumor antigen (not immune receptor) is optimal for direct immune cell engagement applications in the context of engineered universal T cells.

The comparable association signal observed for all covalent compounds in FIG. 9, panel C indicate their comparable ability to form ternary complexes. This suggests the increased CGM efficacy observed in macrophage and NK cell-based immune activation assays is not due to increased ternary complex formation. Moreover, the decreased function associated with the high affinity BM, suggests in the context of inducing cell-cell proximity, even high affinity non-covalent bifunctionals may be limited by mechanical and/or steric strain on ternary complexes. These forces can also potentially induce negative binding cooperativity in cell-cell ternary complex formation. The mechanical and steric strain associated with forming ternary complexes that bridge cells directly may represent a broader challenge to proximity inducing molecules that lack glue function. These systems uniquely benefit from the covalent “glue mimic” approach described in this application, designed for broad applicability to irreversibly stabilize any biologically relevant protein-protein interaction.

### Methods

### Peptide Synthesis and Purification.

All chemical reagents and solvents were obtained from commercial suppliers (Sigma Aldrich or Chem-Impex) and used without further purification. Peptides were synthesized using Standard Fmoc SPPS chemistry. Briefly, Fmoc-Lys-DDe was used to orthogonally insert the SuFEX electrophile (3-fluorosulfonyl benzoic acid), at the desired position on the bifunctional using the different reaction conditions shown in FIG. 2.

All bifunctional constructs, shown in Table 1, were constructed using a Liberty Blue synthesizer. Deprotection was done using 0.1M Oxyma, 20% Piperidine in DMF, for 1 minute at 90 degrees. Coupling was done using IM Oxyma and DIC, with 0.2M Fmoc protected amino acids, in DMF, for 2 minutes at 90 degrees. The benzoic aryl sulfonyl fluoride was coupled at the Nterminus on bead using standard Fmoc amide coupling chemistry. DNP was incorporated as a pre-synthesized Fmoc-Lys (DNP) building block (Chem Impex Cat. #05734), and directly incorporated into the SPPS workflow. When necessary Fmoc-Lys-DDe was used to insert the uPAR labeling SuFEx electrophile. Lys-DDe was deprotected using 15 mL of 2% hydrazide for 15 minutes to orthogonally deprotect the DDe on bead. The benzoic aryl sulfonyl fluoride was then coupled using the free amine. Cleavage from the resin was accomplished using 95/2.5/2.5 TFA: H2O: TIPS for 3 hours. Immediately after cleavage, HPLC and LCMS was conducted to purify and characterize the compound. LCMS data was obtained using an LTQ Orbitrap XL system with a gradient of 95:5 to 5:95 (Water, 0.1% formic acid: Acetonitrile, 0.1% formic acid). A ThermoFisher DIONEX™ UltiMate 3000 UHPLC+was used for HPLC purification with a gradient of 90:10 to 10:90 (Water, 0.1% formic acid: Acetonitrile, 0.1% formic acid). This was then followed by lyophilization.

Ternary complex assessment of AE105-DNP vs BM. Streptavidin probes were placed in 200 μL of 1× Kinetics Buffer, to pre-wet for ten minutes. Next, the probes were placed in 1× Kinetics Buffer for 120 seconds to establish a baseline. This was followed by placing the probes in 75 nM uPAR-Biotin from Acro Biosystems, for 3 minutes. The probes were placed in 1× Kinetics Buffer for 180 seconds in order to re-establish a baseline. 500 nM BM and AE105-DNP was incubated with 250 nM Anti-DNP antibody (Ab) resulting in an increase in association signal with time. Anti-DNP Ab was synthesized as reported previously.⁹ Next, probes coated in antibody ternary complexes were submerged in 1× Kinetics Buffer for 300 seconds in order to measure dissociation kinetics.

Biolayer Interferometry (BLI) Assays on Octet. All Octet Assays were conducted using an Octet Red96 (Sartorius). A volume of 200 AL of each solution was loaded onto a black flat-bottom 96-well plate (Grenier). Probes were always pre-wet for 10 minutes in 1× Kinetics Buffer (Sartorius) prior to experiment.

For ^{uPAR}cGM kinetics, 300 nM of ^{uPAR}cGM was incubated with 75 nM uPAR-Biotin from Acro Biosystems (Cat. No. UPR-H82E7) for various amounts of time. At the end of the incubation time a final concentration of 10 μM of AE105 uPAR competitor was used to quench the reaction. Streptavidin probes were placed in 1× Kinetics Buffer for 2 minutes to establish a baseline. This was followed by placing the probes in the timed incubation solution above, for 3 minutes. The probes were placed in 1× Kinetics Buffer for 3 minutes in order to re-establish a baseline. Next, they were added to a 250 nM Anti-DNP solution in 1× Kinetics Buffer for 5 minutes to measure the association.

For ^{Ab}cBM kinetics, 300 nM of ^{Ab}cBM was incubated with 75 nM of Anti-DNP Ab for various amounts of time. 200 μM of DNP-Glycine competitor was used to quench the reaction. ProG probes (Sartorius) were placed in 1× Kinetics Buffer for 2 minutes to establish a baseline. This was followed by submerging the probes in the timed incubation solution above, for 3 minutes. The probes were then placed in 1× Kinetics Buffer for 3 minutes to re-establish a baseline. Next, they were added to the 500 nM uPAR solution in 1× Kinetics Buffer for 5 minutes to measure the association.

For ^{uPAR}cBM derivative kinetics, the above experiment was repeated, except the incubation time was 2 hours.

For ^{Ab}cBM terminal protein selectivity, 250 nM ^{Ab}cBM was incubated with 100 nM uPAR-biotin overnight. Streptavidin probes were placed in IX Kinetics Buffer for 2 minutes to establish a baseline. This was followed by placing the probes in the incubated solution above, for 3 minutes. The probes were placed in 1× Kinetics Buffer for 3 minutes in order to re-establish a baseline. Next, they were added to a 250 nM anti-DNP solution to measure association.

For ^{uPAR}cBM terminal protein selectivity, ^{uPAR}cBM and ^{uPAR}cBM-2 was incubated with 250 nM Ab overnight. The next day 200 μM DNP-glycine competitor was added to quench the reaction and disrupt any non-covalent complex. Streptavidin probes were placed in 200 μL of IX Kinetics Buffer, to pre-wet for ten minutes. Next, the probes were placed in 1× Kinetics Buffer for 2 minutes to establish a baseline. This was followed by submerging the probes in a 100 nM solution of uPAR-Biotin from Acro Biosystems for 3 minutes. The probes were then placed in 1× Kinetics Buffer for 3 minutes in order to re-establish a baseline. Next, they were added to the 500 nM bifunctional molecule+250 nM Anti-DNP+200 μM DNP-Glycine solution in 1× Kinetics Buffer for 5 minutes to measure the association.

For dual covalent reaction labeling, 100 nM bifunctional molecule was incubated with 100 nM uPAR biotin+50 nM Anti-DNP overnight. The next day Streptavidin probes were placed in 1× Kinetics Buffer for 120 seconds to establish a baseline. This was followed by placing the probes in the incubated solutions above, for 3 minutes. The probes were then placed in 1× Kinetics Buffer with 10 μM AE105 competitor for 4 minutes. Next, they were added to a 50 AM DNP competitor solution for 5 minutes.

^{Ab}cBM and ^{uPAR}cBM selectivity controls on BLI. ^{Ab}cBM selectivity for anti-DNP: 500 nM of ^{uPAR}cBM or ^{Ab}cBM were incubated with 250 nM Isotype IgG (Ab) overnight. The next day streptavidin probes were placed in 200 μL of 1× Kinetics Buffer, to pre-wet for ten minutes. Next, the probes were submerged in 1× Kinetics Buffer solution for 2 minutes to establish a baseline. This was followed by submerging the probes in a solution of 75 nM uPAR-biotin from Acro Biosystems for 3 minutes. Next, the probes were submerged in 1× Kinetics Buffer solution for 2 minutes to re-establish a baseline. Finally, the probes were submerged in the ^{uPAR}cBM or ^{Ab}cBM+isotype Ab solution to measure association.

^{uPAR}cBM selectivity to uPAR: 300 nM of ^{uPAR}cBM or ^{Ab}cBM were incubated with 75 nM BSA-biotin overnight. The next day streptavidin probes were placed in 200 μL of 1× Kinetics Buffer, to pre-wet for ten minutes. Next, the probes were submerged in 1× Kinetics Buffer solution for 2 minutes to establish a baseline. This was followed by submerging the probes in the solution of BSA-Biotin with ^{uPAR}cBM or ^{Ab}cBM. Next, the probes were submerged in 1× Kinetics Buffer solution for 2 minutes to re-establish a baseline. Finally, the probes were submerged in a 250 nM solution of anti-DNP Ab to measure association.

MALDI confirmation of ^{Ab}cBM and ^{uPAR}cBM covalent binary complexes. 10 μL solutions of anti-DNP Ab or uPAR were prepared in 10 mM HEPES buffer, at a 2 mg/mL concentration. 10 μL of 20 μM ^{Ab}cBM, ^{uPAR}cBM, or ^{uPAR}cBM-2 were added to the protein solution and incubated for 12 h. Samples were analyzed by MALDI-TOF using a Bruker Ultraflextreme system. A saturated solution of Sinapic acid was prepared in a 30:70 [v/v] acetonitrile: 0.1% TFA/water solution. The samples were mixed at a 1:1 ratio with the solution. 1 μL was spotted on the plate with BSA used as an external standard. Bruker FlexAnalysis software was used to process data.

Fluorescent SDS-PAGE. Invitrogen Novex™ Wedge Well™ 4 to 20%, 1.0 mm, Tris-Glycine Mini Protein Gels (Catalog #XP04200BOX) were used for gel kinetics assays. In these assays 3 μM Human uPAR or 1.5 μM anti-DNP antibody was mixed with 30 μM CGM-Azide for various times. Reactions were quenched with 15 μM DNP-glycine and incubated with 20 μM DBCO-AF647 for 20 minutes prior to running the gel. Samples were mixed with BioRad™ 4× Laemmli sample buffer (Catalog #161-0747) and were heated at 95° C. for 5 minutes prior to loading on the gel. Samples were run in an Invitrogen mini gel tank at 90 V for the length of the stacking gel and 120 V for the remainder of the resolving gel. Fluorescence was imaged on an Amersham Typhoon™ (Cy5 channel, 360 PMT). The gel was then stained with Bio-Safe Coomassie G-250 Stain (Bio-Rad, Catalog #1610786), the protein contents were visualized using the Odyssey CLX imager. ImageJ software was then used for Densitometry analysis.

SDS-PAGE. For each gel, 4 to 20%, Tris-Glycine, 1.0 mm, Mini Protein Gels from Invitrogen (Catalog #XP04202BOX) were used. 2 μM uPAR was mixed with 0.6 UM Anti-DNP Ab, and with 2.5 μM bifunctional molecule overnight. All samples were mixed with BioRad 4× Laemmli sample buffer (Catalog #161-0747) and heated at 95° C. for 2 minutes prior to loading on the gel. All gels were run in an Invitrogen mini gel tank at 90 V for the length of the stacking gel and 120 V for the length of the resolving gel. Once stained with EZ-Blue Gel Staining solution (Sigma) the protein contents were visualized using a 700 nM laser on an Odyssey CLX imager. For Densitometry analysis ImageJ software was used.

Determining covalent ternary complex formation kinetics using SDS-PAGE assays. SDS-PAGE gels were run as described above. 2 μM uPAR, was mixed with 0.6 UM anti-DNP Ab and 2.5 μM CGM, for various times (0 to 24 hours).

Cell Culture. All flow cytometry experiments were run on a Beckman Coulter CytoFLEX™ Flow Cytometer or a BD LSR II. The human IgG isotype control used was purchased from Jackson ImmunoResearch (Cat. No. 009-000-003). For uPAR selectivity controls, AE133 competitor peptide lacking the DNP moiety was used. A172 cells were purchased from ATCC (Cat. No. CRL-1620). U937 cells were generously given by Dr. John Valliant (McMaster University, Canada). IFN-γ was purchased from Fischer Scientific (PHC4031). Ultra low IgG FBS was purchased from Fischer Scientific (A3381901). DiD cell dye was purchased from Fischer Scientific (V22887). 96-Well U-bottom plates were purchased from Fischer Scientific (08-772-17). Pen/Strep was purchased from Fischer Scientific (15140-122). FBS was purchased from Fischer Scientific (12484-028).

uPA-uPAR Competition experiment via widefield microscopy. A 20:1 molar ratio of BroadPharm Fluor 647 NHS Ester (Catalog #BP-24069) was mixed with 25AM Human uPa Protein (Acro Biosystems, PLU-H5229) at room temperature for 1 hour. To purify the protein conjugates, NAb Protein A Plus Spin Columns, 0.2 mL (Thermo Scientific) were used. A final concentration of 20.75 μM of uPa-AF647 was calculated using a NanoDrop UV-Vis Spectrophotometer (Thermo Scientific). uPAR+A172 cells were lifted using 0.5% Trypsin-EDTA (Gibco) and quenched with complete growth media. 100 μL of cells were seeded at a concentration 50 000 cells/mL. 50 μL of 100 nM CGM or BM were added to cells for 15 minutes prior to washing the cells and adding 100 μL of 25 nM of uPa-AF647. Samples were washed again and imaged on a Zeiss Celldiscoverer⁷ inverted widefield microscope, using a Plan-Apochromat 20x/0.7 NA lens with a Colibri light source. uPa-AF647 was detected using a 631/33 nm excitation filter and a 687/145 nm emission filter. The nuclei were stained using DAPI, detected using a 385/30 nm excitation wavelength, and a 425/30 nm emission filter. The DAPI and the AF647 Channel were at 10.00% and 27.50% LED power, respectively. The sample was imaged with an Axiocam™ 712 mono camera, exposure time was set to 55 ms and 175 ms for the DAPI and AF647 channel, respectively. Images were acquired using ZenBlue software.

Cell surface Covalent Ternary Complex Crosslinking. A 20:1 molar ratio of BroadPharm Fluor 647 NHS Ester (Catalog #BP-24069) was mixed with 12.25 μM Anti-DNP Ab at room temperature for 1 hour. NAb Protein A Plus Spin Columns, 0.2 mL (Thermo Scientific) were used to remove free dye. A final concentration of 1.23 mg/mL of SPE7-AF647 was calculated using a NanoDrop UV-Vis Spectrophotometer (Thermo Scientific). uPAR+/−A172 cells were suspended using 1 mM EDTA and quenched with complete growth media. The cells were seeded at a concentration of 1,500,000 cells/mL using serum free assay media (neat DMEM). To a U-bottom 96-well plate (Fischer Scientific V22887), 100 μL of cells were added to 1 μM of CGM or BM and 500 nM SPE7-AF647 and placed in a 37° C. 5% CO₂ incubator for 4 hours. All samples were washed three times with serum free assay media (neat DMEM) and plates were placed on ice before running flow cytometry. The following gains were used, FSC: 81 SSC: 109 APC: 200

Antibody dependent cell mediated phagocytosis (ADCP) assay. For preparation of effector monocytes, 24 hours prior to inducing phagocytosis, U937 monocytes were seeded at 5×10⁵ cells/mL and activated with IFN-γ (0.1 mg/m.L). After incubation, cells were counted and washed twice with serum free assay media (neat DMEM). Cells were then suspended to a concentration of 1 million cells/mL and stained with 1.9 μM Vybrant™ DiD Cell-Labeling Solution for 30 minutes (37° C., 5% CO₂). Cells were then washed 3× with warm assay media (AM, 14% Ultra Low IgG FBS in DMEM) and resuspended to a concentration of 3.0×106 million cells. The day prior, 100 nM Biotin-uPAR+200 nM bifunctional molecule+100n.M anti-DNP Ab was incubated overnight. The next day 10 million Streptavidin YG beads (Polysciences Cat. #24157) were washed 2× with PBS. The uPAR biotin, anti-DNP Ab, and bifunctional molecule solution was then incubated with 1.5×105 Streptavidin beads for 1 hour. Next, the beads were washed and combined with 1.5×105 monocytes. Next, the solutions were spun down at 800 RPM and incubated at 37 degrees for 1 hour. This was followed by placing solutions on ice to end phagocytosis, prior to flow cytometry analysis. Quadrant 1 represents target bead population only. Quadrant 2 represents macrophage engulfed target bead population. ADCP percentage was calculated by dividing Q2/(Q2+Q1)×100%. The following voltages were used: FSC: 330, SSC: 270, PE: 310, and AF647: 400.

Macrophage antibody dependent cell mediated phagocytosis (ADCP) selectivity controls. ADCP experiments were run as described above. To probe selectivity, 125 AM of DNP-glycine competitor, or 30 μM AE133 competitor was placed into experimental conditions. Alternatively, isotype control antibody was used instead of anti-DNP. Q1: Top left is beads only population. Q2: Top right is double positive monocytes with phagocytosed target bead population. ADCP percentage was calculated by dividing Q2/(Q2+Q1)×100%.

Model NK Cell CD16 Activation assay (ADCC) with selectivity controls. ADCC was quantified using the ADCC Reporter Bioassay kit (Promega G7010), as described above. suPAR KO is a A172 CRISPR-CAS9 knockout of uPAR, and was used to probe selectivity to uPAR. Isotype IgG was used to probe selectivity to anti-DNP Ab.

CD16α Activation Assay. ADCC was quantified using the ADCC Reporter Bioassay kit (Promega G7010). Target cells were seeded at a density of 2.5×10⁴ cells per well in opaque 96 well flat-bottom plates (Corning Costar, 3917) in complete media. Sixteen hours after seeding, cells were washed gently with 100 μL of neat RPMI. To the cells, 25 μL of RPMI supplemented with 4% ultra-low IgG FBS was added followed by 25 μL of a dilution series starting at 250 nM of BM, ^{Ab}cBM, ^{uPAR}cBM, or CGM with 125 nM antibody. After a 45-minute incubation, 2 μL of supplemented RPMI containing 7.5×104 Jurkat effector cells expressing human CD16 were added to each well. The plates were then incubated for 24 additional hours. 75 μL of Bio-Glo Luciferase Assay Reagent was added to each well, and luminescence was quantified using the SpectraMax i3 plate reader.

Receptor generation and gamma retrovirus production for SAR-T Cell System. The SPE7 variable heavy-variable light (VHVL) scFv was first designed using the crystal structure (PDB: 10AU) and synthesized by Genescript. Human CD8α signal peptide was used. The anti-DNP scFv containing engineered receptors were cloned into the pRV100G backbone. Gammaretrovirus was generated to be used for subsequent engineering of T-cells. Briefly, PLAT-E cells were first transduced with the anti-DNP SAR containing plasmids (15 μg) and pCl Eco (15 μg) using using Opti-MEM (Gibco) and Lipofectamine 2000 (Thermo Fisher Scientific). Ecotropic gamma retrovirus was concentrated by centrifugation in Amicon filter system (Millipore™ Sigma) and stored at −80° C. This virus was then used to transduce PG-13 cells. After 3 days of consecutive transduction, the PG13s were scaled up and subsequently the virus containing supernatant was filtered through a 0.45 μm filter (Brand) and stored at −80° C. to be used for T cell engineering.

Engineering of human T cells. Peripheral blood mononuclear cells (PBMCs) were obtained from healthy donors. In some cases, PBMC were collected from commercial leukapheresis products (HemaCare and StemCell Technologies). PBMCs were isolated by Ficoll-Paque-Plus gradient centrifugation (GE Healthcare) and cryopreserved in inactivated human AB serum (Corning), containing 10% DMSO (Sigma-Aldrich), or Cryostor™ CS10 (Stemcell). To produce primary human αβ T cells, 1×106 PBMCs were seeded in a 24-well plate and stimulated with ImmunoCult™ CD3/CD28/CD2 soluble activator (Stemcell) at a concentration of 25 μL/mL and cultured in RPMI 1640 containing 10% FBS, 10 mM HEPES (Roche Diagnostics), 1 mM sodium pyruvate (Sigma-Aldrich), 1 mM non-essential amino acids (Gibco), 55 μM β-mercaptoethanol (Gibco), 100 U/mL penicillin (Gibco), 100 μg/mL streptomycin (Gibco), 100 I.U./mL rhL-2 and 10 ng/mL rhIL-7 (PeproTech). 48 hours later, cells were transferred to a 24-well non tissue culture coated plate (Falcon) that was pre-coated with retronectin (10 μg/mL) and anti-DNP scFv SAR gamma retrovirus for transduction. 24 hours later 1 mL of T-cell media supplemented with rhL-2 (100 I.U./mL,1.5 ng/ml) and rhIL-7 (10 ng/mL) (PeproTech) is added to each well. 48 hours after the media addition the cells are washed with PBS and scaled into a larger vessel. Cells were cultured for a total period of 14 days prior to cryopreservation. T cells were cryopreserved in Cryostor CS10 (StemCell Technologies) according to manufacturer's instructions.

Synthetic Antigen Receptor (SAR) T-cell proliferation assay. Anti-DNP KIR-CAR engineered T cells (5×10⁵ cells) labeled with CellTrace Violet dye (Cat No. C34557 Invitrogen) were incubated with ^{Ab}cBM, ^{uPAR}cBM, or CGM, and with A172 eGFP expressing tumor targets at an E:T ratio of 1:1 or left unstimulated in media. All proliferation assay samples were incubated for 3 days at 37° C. and 5% CO₂. Cells were then stained with Live/Dead Fixable Near-IR stain (Cat No. L10119, Invitrogen), PerCP-Cy5.5-conjugated mouse anti-human CD8α (Cat No. 45-0088-42, eBioscience), Alexa Fluor 700-conjugated mouse anti-human CD4 (Cat No. 56-0048-82, eBioscience), VioBright FITC-conjugated mouse anti-human NGFR (Cat No. 130-113-423, Miltenyi Biotec), and BV605-conjugated mouse anti-human CD3 (Cat No. 300460 BioLegend). Results were analyzed with FCS Express (De Novo Software) by determining the starting generation peak based on the unstimulated sample and using the software proliferation package for fitting a proliferation model and collecting corresponding statistics, such as percent divided. FlowJo analysis software was used generate histogram CTV dilution curves. CD4 and C8 T cells showed the same results, only CD8 T cells are displayed here.

Live Cell Killing Assay with SAR-T Cell System. A172 glioblastoma cells were engineered with eGFP lentivirus made in house. In these experiments, 5×103 tumor cells per well were plated in a 96 well flatbottom plate overnight. The next day anti-DNP SAR αβ T cells were added to the tumor cells at an effector to target ratio of 8:1. Also added to the wells were the bifunctionals at various concentrations (InM, 10 nM, 100 nM). The 3 components were co-cultured for 3 days at 37° C. and 5% CO₂ in the Sartorius Incucyte™ S3 Live cell imaging system with 9 images per well taken every 8 hours. For the A172 eGFP cells, the green image mean for each image was used to determine tumor cell growth. The area under the growth curve (AUC) was analyzed using Prism GraphPad and used as a metric for tumor cell growth for this data. The larger the area, the greater the tumor cell growth that occurred over the incubation period. The area under the curve for the tumor alone control and each condition were used to calculate the % cytotoxicity. Percent cytotoxicity was calculated as: (AUC Tumor Alone-AUC Sample)/(AUC Tumor Alone)×100%

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### CITATIONS

1. Stanton, B. Z., Chory, E. J. & Crabtree, G. R. Chemically induced proximity in biology and medicine. Science (1979) 359, (2018).
2. Gerry, C. J. & Schreiber, S. L. Unifying principles of bifunctional, proximity-inducing small molecules. Nat Chem Biol 16, 369-378 (2020).
3. Michnick, S. W., Rosen, M. K., Wandless, T. J., Karplus, M. & Schreiber, S. L. Solution Structure of FKBP, a Rotamase Enzyme and Receptor for FK506 and Rapamycin. Science (1979) 252, 836-839 (1991).
4. Liu, S. et al. Rational Screening for Cooperativity in Small-Molecule Inducers of Protein-Protein Associations. J Am Chem Soc 145, 23281-23291 (2023).
5. Banaszynski, L. A., Liu, C. W. & Wandless, T. J. Characterization of the FKBP. Rapamycin. FRB Ternary Complex. J Am Chem Soc 127, 4715-4721 (2005).
6. Schreiber, S. L. The Rise of Molecular Glues. Cell 184, 3-9 (2021).
7. Dewey, J. A. et al. Molecular Glue Discovery: Current and Future Approaches. Journal of Medicinal Chemistry vol. 9278-9296 66 Preprint at https://doi.org/10.1021/acs.jmedchem.³c00449 (2023).
8. Lake, B., Serniuck, N., Kapcan, E., Wang, A. & Rullo, A. F. Covalent Immune Recruiters: Tools to Gain Chemical Control over Immune Recognition. ACS Chem Biol 15, 1089-1095 (2020).
9. Kapcan, E. et al. Covalent Stabilization of Antibody Recruitment Enhances Immune Recognition of Cancer Targets. Biochemistry (2021) doi: 10.1021/acs.biochem.¹c00127.
10. Guo, S., Efremov, A. K. & Yan, J. Understanding the catch-bond kinetics of biomolecules on a one-dimensional energy landscape. Commun Chem 2, (2019).
11. Rakshit, S., Zhang, Y., Manibog, K., Shafraz, O. & Sivasankar, S. Ideal, catch, and slip bonds in cadherin adhesion. Proc Natl Acad Sci USA 109, 18815-18820 (2012).
12. Kapcan, E. & Rullo, A. F. A covalent opsonization approach to enhance synthetic immunity against viral escape variants. Cell Rep Phys Sci 4, 101258 (2023).
13. Dong, J., Krasnova, L., Finn, M. G. & Sharpless, K. B. Sulfur (VI) Fluoride Exchange (SuFEx): Another Good Reaction for Click Chemistry. Angewandte Chemie International Edition 53, 9430-9448 (2014).
14. McCann, H. M. et al. Covalent Immune Proximity-Induction Strategy Using SuFEx-Engineered Bifunctional Viral Peptides. ACS Chem Biol 17, 1269-1281 (2022).
15. Mack, E. T. et al. Dependence of Avidity on Linker Length for a Bivalent Ligand-Bivalent Receptor Model System. J Am Chem Soc 134, 333-345 (2012).
16. Krishnamurthy, V. M., Semetey, V., Bracher, P. J., Shen, N. & Whitesides, G. M. Dependence of Effective Molarity on Linker Length for an Intramolecular Protein-Ligand System. J Am Chem Soc 129, 1312-1320 (2007).
17. Zheng, Q. et al. SuFEx-enabled, agnostic discovery of covalent inhibitors of human neutrophil elastase. Proceedings of the National Academy of Sciences 116, 18808-18814 (2019).
18. Mortenson, D. E. et al. “Inverse Drug Discovery” Strategy To Identify Proteins That Are Targeted by Latent Electrophiles As Exemplified by Aryl Fluorosulfates. J Am Chem Soc 140, 200-210 (2018).
19. Zhai, B. T. et al. Urokinase-type plasminogen activator receptor (uPAR) as a therapeutic target in cancer. J Transl Med 20, 1-24 (2022).
20. Ploug, M. et al. Peptide-derived antagonists of the urokinase receptor. Affinity maturation by combinatorial chemistry, identification of functional epitopes, and inhibitory effect on cancer cell intravasation. Biochemistry 40, 12157-12168 (2001).
21. Llinas, P. et al. Crystal structure of the human urokinase plasminogen activator receptor bound to an antagonist peptide. EMBO Journal 24, 1655-1663 (2005).
22. Jenkins, M. R., Tsun, A., Stinchcombe, J. C. & Griffiths, G. M. The Strength of T Cell Receptor Signal Controls the Polarization of Cytotoxic Machinery to the Immunological Synapse. Immunity 31, 621-631 (2009).
23. Lallemand, C. et al. A Novel System for the Quantification of the ADCC Activity of Therapeutic Antibodies. J Immunol Res 2017, U.S. Pat. No. 3,908,289 (2017).
24. Minutolo, N. G. et al. Quantitative Control of Gene-Engineered T-Cell Activity through the Covalent Attachment of Targeting Ligands to a Universal Immune Receptor. J Am Chem Soc 142, 6554-6568 (2020).
25. Ruffo, E. et al. Post-translational covalent assembly of CAR and synNotch receptors for programmable antigen targeting. Nat Commun 14, (2023).
26. Kim, M. S. et al. Redirection of genetically engineered CAR-T cells using bifunctional small molecules. J Am Chem Soc 137, 2832-2835 (2015).

Sequences

Native CP33 peptide sequence (SEQ ID NO: 1):
VNSCLLLPNLLGCGDD
Disulfide bond between C4 and C13
C-terminal amidation (-CONH₂) may be present or absent
CP33 peptide sequence variant (SEQ ID NO: 2):
VNSCLLLPNLLGCDGD
Disulfide bond between C4 and C13
C-terminal amidation present or absent
CP33 C-term tyrosine peptide sequence (SEQ ID NO: 3):
XVNSCLLLPNLLGCGDDY
X is K, Ac-K(azido), or absent
Disulfide bond between C5 and C14
C-terminal amidation present or absent
CP33 C-term tyrosine peptide sequence (SEQ ID NO: 4):
XVNSCLLLPNLLGCDGDY
X is K, Ac-K(azido), or absent
Disulfide bond between C5 and C14
C-terminal amidation present or absent
CP33 internal tyrosine (G13Y) mutant peptide sequence (SEQ ID NO: 5):
XVNSCLLLPNLLYCGDD
X is K, Ac-K(azido), or absent
Disulfide bond between C5 and C14
C-terminal amidation present or absent
CP33 internal tyrosine (G13Y) mutant peptide sequence (SEQ ID NO: 6):
XVNSCLLLPNLLYCDGD
X is K, Ac-K(azido), or absent
Disulfide bond between C5 and C14
C-terminal amidation present or absent
CP33 N-term tyrosine peptide sequence (SEQ ID NO: 7):
XGYVNSCLLLPNLLGCGDD
X is K, Ac-K(azido), or absent
Disulfide bond between C7 and C16
C-terminal amidation present or absent
CP33 N-term tyrosine peptide sequence (SEQ ID NO: 8):
XGYVNSCLLLPNLLGCDGD
X is K, Ac-K(azido), or absent
Disulfide bond between C7 and C16
C-terminal amidation present or absent
CP33-nt-aryl SuFEx peptide sequence (SEQ ID NO: 9):
XVNSCLLLPNLLGCGDD
X is K or Aryl-SuFEx cap-K(azido)
Disulfide bond between C5 and C14
C-terminal amidation present or absent
CP33-nt-aryl SuFEx peptide sequence (SEQ ID NO: 10):
XVNSCLLLPNLLGCDGD
X is K or Aryl-SuFEx cap-K(azido)
Disulfide bond between C5 and C14
C-terminal amidation present or absent
CP33-ct-aryl SuFEx peptide sequence (SEQ ID NO: 11):
XVNSCLLLPNLLGCGDDX
X1 is K or K(azido)
X18 is K or K-SuFEx
Disulfide bond between C5 and C14
CP33-ct-aryl SuFEx peptide sequence (SEQ ID NO: 12):
XVNSCLLLPNLLGCDGDX
X1 is K or K(azido)
X18 is K or K-SuFEx
Disulfide bond between C5 and C14
Synthetic uPAR binding peptide ligand (SEQ ID NO: 13)
L-Lys-Gly-Gly-L-Ser-Gly-L-Asp-L-Cha-L-Phe-D-Ser-D-Arg-L-Tyr-L-Leu-L-Trp-
L-Ser
Modified synthetic uPAR binding peptide ligand (SEQ ID NO: 14)
L-Lys-Gly-Gly-L-Ser-Gly-L-Asp-L-Cha-L-Phe-D-Ser-D-Arg-L-Ala-L-Leu-L-Trp-L-
Ser

1. A compound of Formula (I), Formula (II) or Formula (III) or a pharmaceutically acceptable salt, and/or solvate thereof, 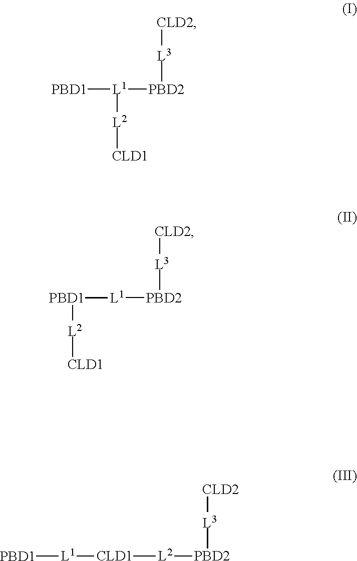
wherein PBD1 and PBD2 are each independently a protein binding domain (PBD) and CLD1 and CLD2 are each independently a covalent labeling domain (CLD), and L1, L2 and L3 are each independently a linker group;
wherein PBD1 selectively binds to a first target protein and PBD2 selectively binds to a second target protein, and wherein CLD1 comprises a functional group that, upon binding of PBD1 to the first target protein, forms an irreversible covalent bond with a nucleophilic group in the first target protein and CLD2 comprises a functional group that, upon binding of PBD2 to the second target protein, forms an irreversible covalent bond with a nucleophilic group in the second target protein.

2. The compound of claim 1, wherein CLD1 and CLD2 each independently comprise an electrophilic functional group that reacts with a nucleophilic moiety on the first and second target proteins.

3. The compound of claim 1, wherein CLD1 and CLD2 each independently comprise a fluorosulfate, fluorosulfonate or sulfonyl fluoride group.

4. The compound of claim 3, wherein the sulfonyl fluoride group has the following structure:

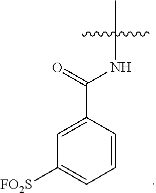

5. The compound of claim 1, wherein the first and second target proteins are each independently selected from: proteins that are cell surface receptors;
proteins that are overexpressed in a disease, disorder or condition;
proteins that are expressed on the surface of a cancer cell;
a tumor antigen; an antibody; and an immune cell receptor.

6. The compound of claim 1, wherein the first and second target proteins are each independently selected from an urokinase plasminogen activating receptor (uPAR), a prostate-specific membrane antigen (PSMA), a human epidermal growth factor receptor 2 (HER2), an integrin, CD38, programmed death-ligand-1 (PD-L1), a G protein-coupled receptor (GPCR), a Kirsten rat sarcoma virus (KRAS), a vascular endothelial growth factor (VEGF) and a folate receptor.

7. The compound of claim 1, wherein PBD1 and PBD2 are different and each independently:
(1) a di- or trinitrophenyl group having the following structure: 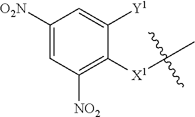
wherein Y1 is H or NO₂;
X¹ is NR¹, O, CH₂, S(O), SO₂, SO₂O, OSO₂ or OSO₂O; and
R¹ is H, C1-4alkyl or C(O)C₁₋₄alkyl;
(2) a bicyclic nitro-substituted aromatic group having the following structure: 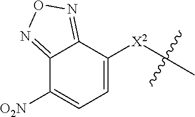
wherein X² is a bond, O, CH₂, NR² or S; and
R² is H, C1-4alkyl or C(O)C₁₋₄alkyl;
(3) a galactose-containing carbohydrate having the following structure: 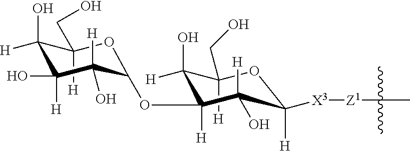
wherein X³ is CH₂, O, NR³ or S;
R³ is H or C1-4alkyl; and
Z¹ is a bond, monosaccharide, disaccharide, oligosaccharide, glycoprotein or glycolipid;
(4) a group having the following structure: 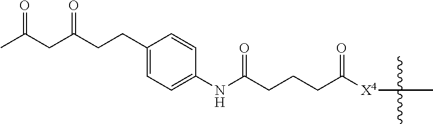
wherein X⁴ is O, CH₂ or NR⁴; and
R⁴ is H, C1-4alkyl or C(O)C₁₋₄alkyl;
(5) a group having the following structure: 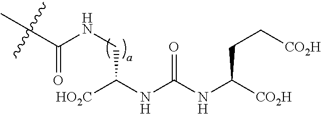
wherein a is an integer from 0 to 10, 1 to 15, 1 to 10, 1 to 8, or 1, 2, 3, 4, 5 or 6;
(6) a group having the following structure: 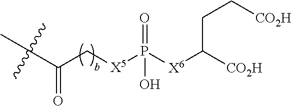
wherein X⁵ and X⁶ are independently CH₂, O, NH or S; and
b is an integer from 0 to 10, 1 to 15, 1 to 10, 1 to 8, or 1, 2, 3, 4, 5 or 6;
(7) a group having the following structure: 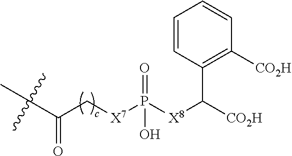
wherein X¹ and X⁸ are independently CH₂, O, NH or S; and
c is an integer from 0 to 10, 1 to 15, 1 to 10, 1 to 8, or 1, 2, 3, 4, 5 or 6;
(8) a group having the following structure: 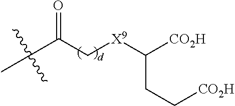
wherein X⁹ is O, CH₂, NR⁵, S(O), SO₂, SO₂O, OSO₂ or OSO₂O;
R⁵ is H, C1-4alkyl or C(O)C₁₋₄alkyl; and
d is an integer from 0 to 10, 1 to 15, 1 to 10, 1 to 8, or 1, 2, 3, 4, 5 or 6;
(9) biotin or a biotin analog having the following structure: 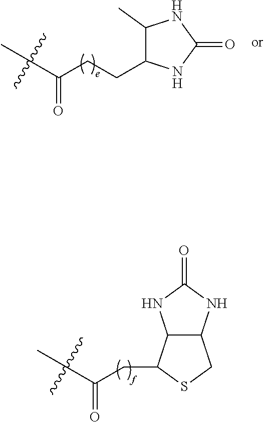
wherein e and f are, independently, an integer from 0 to 10, 1 to 15, 1 to 10, 1 to 8, or 1, 2, 3, 4, 5 or 6;
(10) a circular peptide 33 (CP33) or functional variants thereof having the sequence:
VNSCLLLPNLLGCGDD (SEQ ID NO: 1), wherein C4 and C13 form a disulfide bond,
VNSCLLLPNLLGCDGD (SEQ ID NO: 2), wherein C4 and C13 form a disulfide bond,
SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12,
SEQ ID NO: 3, wherein X is K and wherein C5 and C14 form a disulfide bond,
SEQ ID NO: 4, wherein X is K and wherein C5 and C14 form a disulfide bond,
SEQ ID NO: 7, wherein X is K and wherein C7 and C16 form a disulfide bond,
SEQ ID NO: 8, wherein X is K and wherein C7 and C16 form a disulfide bond,
SEQ ID NO: 9, wherein X is K and wherein C5 and C14 form a disulfide bond,
SEQ ID NO: 10, wherein X is K and wherein C5 and C14 form a disulfide bond,
SEQ ID NO: 11, wherein X¹ is K, wherein X¹⁸ is K, and wherein C5 and C14 form a disulfide bond, or
SEQ ID NO: 12, wherein X¹ is K, wherein X¹⁸ is K, and wherein C5 and C14 form a disulfide bond; or
(11) a synthetic peptide comprising an amino acid sequence of L-Lys-Gly-Gly-L-Ser-Gly-L-Asp-L-Cha-L-Phe-D-Ser-D-Arg-L-Tyr-L-Leu-L-Trp-L-Ser (SEQ ID NO: 13), L-Lys-Gly-Gly-L-Ser-Gly-L-Asp-L-Cha-L-Phe-D-Ser-D-Arg-L-Ala-L-Leu-L-Trp-L-Ser (SEQ ID NO: 14), or functional variants thereof.

8. The compound of claim 1, wherein L1, L2 and L3, are each independently a direct bond, C₁₋₂₀ alkylene, optionally interrupted by triazolyl, piperdinyl, pyrrolidinyl, and/or one or more heteromoieties such as O, S, S(O), SO₂, OSO₂, SO₂O,OSO₂O, NR⁸, C(O), NHC(O), or C(O)NH, wherein R⁸ is H or C1-4alkyl.

9. The compound of claim 1, wherein at least one of the first and second target proteins is an antibody.

10. The compound of claim 1, wherein at least one of the first and second target proteins is an immune cell receptor, and optionally the immune cell is an engineered T cell.

11. The compound of claim 1, wherein:
the first target protein is an antibody and the second target protein is a cell surface receptor;
the first target protein is an antibody and the second target protein is a tumor antigen;
PBD1 comprises a hapten that selectively binds to an antibody comprising a hapten binding site and PBD2 comprises a cell surface receptor binding domain;
PBD1 comprises a hapten that selectively binds to an antibody comprising a hapten binding site and PBD2 comprises a tumor antigen binding domain;
the first and second target proteins are each a cell surface receptor;
the PBD1 selectively binds to an immune cell receptor and PBD2 selectively binds to a cell surface receptor; or
PBD1 selectively binds to an immune cell receptor and PBD2 selectively binds to a tumor antigen.

12. The compound of claim 1, wherein the compound is selected from: 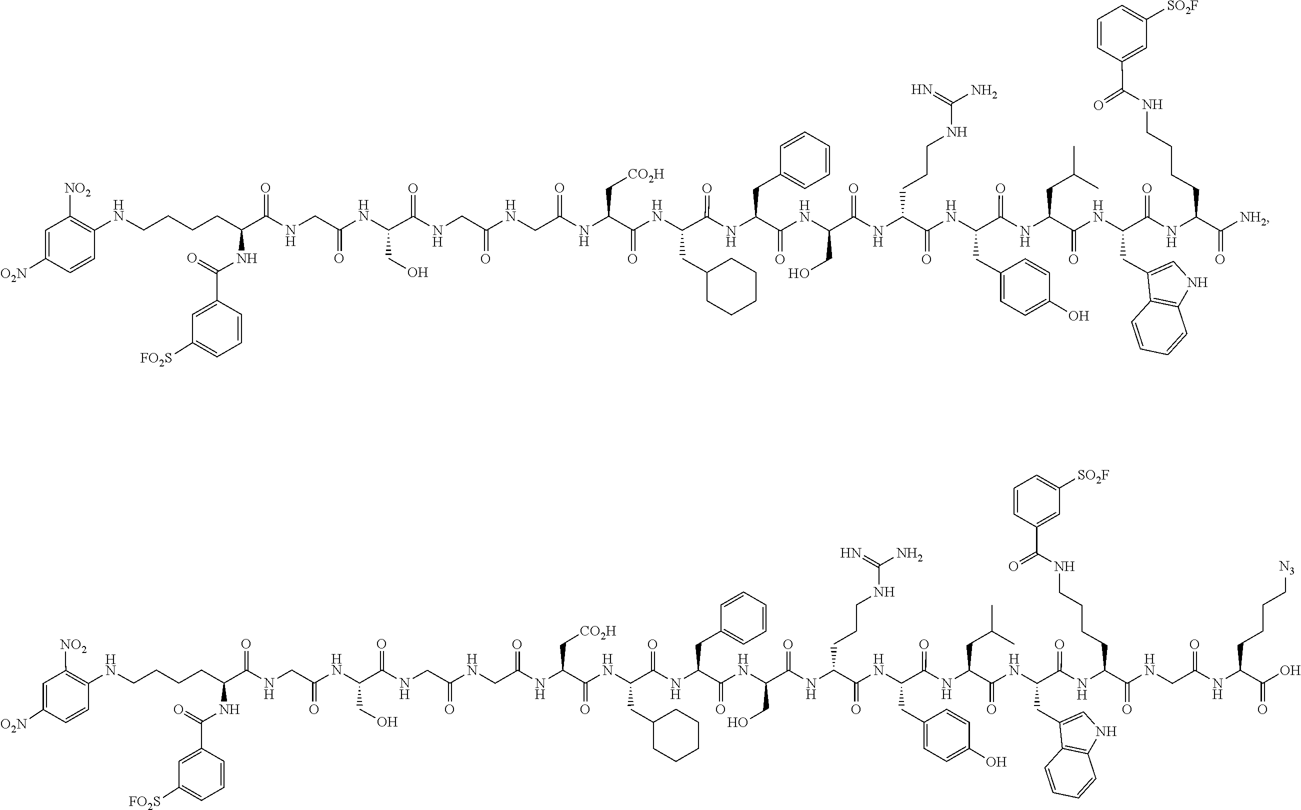
and a pharmaceutically acceptable salt and/or solvate thereof.

13. A composition comprising the compound of Formula (I), Formula (II) or Formula (III), or a pharmaceutically acceptable salt and/or solvate thereof, of claim 1 and at least one carrier, diluent and/or excipient.

14. A method for forming ternary protein complexes, either in a biological sample or in a subject, comprising administering an effective amount of a compound of Formula (I), Formula (II) or Formula (III), or a pharmaceutically acceptable salt and/or solvate thereof, of claim 1 to the biological sample or subject.

15. A method for recruiting an antibody or an immune cell for immunotherapy, either in a biological sample or in a subject, comprising administering an effective amount of a compound of Formula (I), Formula (II) or Formula (III), or a pharmaceutically acceptable salt and/or solvate thereof, of claim 1 to the biological sample or subject.

16. A method for recruiting an antibody or an immune cell and targeting a cell for provoking an immune response to the cell, either in a biological sample or in a subject, comprising administering an effective amount of a compound of Formula (I), Formula (II) or Formula (III), of Formula (I), Formula (II) or Formula (III), or a pharmaceutically acceptable salt and/or solvate thereof, of claim 1 to the biological sample or the subject.

17. A method for binding tumor antigens on a cell, either in a biological sample or in a subject, comprising administering an effective amount of a compound of Formula (I), Formula (II) or Formula (III), of Formula (I), Formula (II) or Formula (III), or a pharmaceutically acceptable salt and/or solvate thereof, of claim 1 to the biological sample or the subject.

18. A method of treating a disease, disorder, or condition that is treatable by engaging an immune response, comprising administering a therapeutically effective amount of a compound of Formula (I), Formula (II) or Formula (III), of Formula (I), Formula (II) or Formula (III), or a pharmaceutically acceptable salt and/or solvate thereof, of claim 1 to a subject in need thereof.

19. The method of claim 18, wherein the disease, disorder, or condition treatable by engaging an immune response is cancer, or the disease, disorder, or condition is selected from an autoimmune disease, allergy and transplant rejection.

20. The method of claim 18, wherein administering the compound or composition comprises first exposing the compound or composition to the first target protein ex vivo, so that the PBD1 binds its target protein and a covalent bond between the target protein and the compound is formed and then administering the resulting complex to the biological sample or subject, whereby the resulting complex then binds via the PBD2 to its target.
